(19) United States
(12) Patent Application Publication
(10) Pub. No.: US 2018/0221289 A1
(43) Pub. Date: Aug. 9, 2018

(54) PHOSPHOLIPID-CHOLESTERYL ESTER NANOFORMULATIONS AND RELATED METHODS

(71) Applicant: Autotelic LLC, City of Industry, CA (US)

(72) Inventor: Vuong Trieu, Agoura Hills, CA (US)

(73) Assignee: Autotelic LLC, City of Industry, CA (US)

(21) Appl. No.: 15/750,764

(22) PCT Filed: Aug. 8, 2016
(86) PCT No.: PCT/US16/46064

§ 371 Date: Feb. 6, 2018

Related U.S. Application Data
(60) Provisional application No. 62/202,036, filed on Aug. 6, 2015.
(60) Provisional application No. 62/350,592, filed on Jun. 15, 2016.

(51) Int. Cl. A61K 9/50; A61K 31/337; A61P 35/00; A61K 9/00; A61K 9/51; A61P 9/10

(52) U.S. Cl. CPC A61K 9/5015; A61K 31/337; A61P 35/00; A61P 9/10; A61K 9/5169; A61K 9/5123; A61K 9/0019

(57) ABSTRACT

In one aspect, the invention provides therapeutic agent nanoparticles coated with cholesteryl esters, formulations of the nanoparticles suitable for injection, methods for administering therapeutic agents and for treating diseases and conditions treatable by the therapeutic agents using the formulations. In a related aspect, the invention provides synthetic high density lipoprotein nanoparticles useful for therapeutic agent delivery, and methods for their preparation and use.

### CROSS-REFERENCES TO RELATED APPLICATIONS

This application claims the benefit of U.S. Application No. 62/202,036, filed Aug. 6, 2015, and U.S. Application No. 62/350,592, filed Jun. 15, 2016, each expressly incorporated herein by reference in its entirety.

### BACKGROUND OF THE INVENTION

The effective delivery of hydrophobic therapeutic agents remains a challenging problem for the pharmaceutical industry. These challenges relate to the difficulty in formulating these therapeutic agents in vehicles for administration. Historically, hydrophobic therapeutic agents are administered in delivery vehicles that are less than advantageous with regard to delivery properties including therapeutic agent dose and bioavailability. Furthermore, serious side effects are occasionally observed associated with the vehicle itself.

The formulation of paclitaxel over the years is an example of the challenges associated with many hydrophobic therapeutic agents.

Paclitaxel is one of the most effective chemotherapeutic drugs and is used to treat mainly breast, lung, and ovarian cancers. Taxol® is a paclitaxel formulation that utilizes a solvent, cremophor EL, to solubilize and deliver the essentially water-insoluble paclitaxel. Disadvantages and side effects of Taxol® are directly associated this solvent.

Paclitaxel has also been formulated as nanoparticles. Abraxane® is a nanoparticle paclitaxel formulation having improved paclitaxel solubility (0.35-0.7 μg/mL) compared to Taxol® and avoids the use of a harmful solvent. Abraxane® is a human serum albumin-coated paclitaxel nanoparticle. Cynviloq®, a polymeric micelle paclitaxel formulation that uses a biocompatible chemical polymer rather that a biological polymer to stabilize the nanoparticle, is a next-generation paclitaxel product.

Despite the advances in the development of alternative formulations that overcome the disadvantages associated with known hydrophobic therapeutic agent formulations, a need exists for new formulations of hydrophobic therapeutic agents having improved properties. The present invention seeks to fulfill these needs and provides further related advantages.

### SUMMARY OF THE INVENTION

The present invention provides therapeutic agent nanoparticles, nanoparticle formulations suitable for injection, methods for administering therapeutic agents and for treating diseases and conditions treatable by the therapeutic agents using the formulations.

In one aspect, the invention provides phospholipid-coated therapeutic agent nanoparticles.

In one embodiment, the phospholipid-coated therapeutic agent nanoparticle comprises a particulate therapeutic agent coated with a cholesteryl ester and one or more phospholipids, wherein the nanoparticle is stable in aqueous delivery vehicles for administration and releases the therapeutic agent substantially instantaneously upon exposure to physiological fluid. In certain of these embodiments, the nanoparticle is as stable in aqueous delivery vehicles for administration as synthetic polymeric micelles containing a therapeutic agent (Genexol-PM®) and is as effective in releasing the therapeutic agent under physiological conditions as a human-serum albumin-coated therapeutic agent (Abraxane®).

In another embodiment, the phospholipid-coated therapeutic agent nanoparticle comprises a particulate therapeutic agent coated with a cholesteryl ester and one or more phospholipids, wherein the phospholipid is selected from the group consisting of a mono-acylphospholipid, a diacylphospholipid, and mixtures thereof.

In a further embodiment, the phospholipid-coated therapeutic agent nanoparticle consists essentially of a particulate therapeutic agent coated with a cholesteryl ester and one or more phospholipids.

In yet a further embodiment, the phospholipid-coated therapeutic agent nanoparticle consists of a particulate therapeutic agent coated with a cholesteryl ester and one or more phospholipids.

In certain of the above embodiments, the cholesteryl ester has a fatty acid component having from 10 to 22 carbons. In other of these embodiments, the cholesteryl ester has a fatty acid component having from 12 to 20 carbons. In further of these embodiments, the cholesteryl ester has a fatty acid component having from 16 to 18 carbons.

In certain of the above embodiments, the ratio of cholesteryl ester to phospholipid is from about 1:1000 to about 1:10 w/w. In other of these embodiments, the ratio of cholesteryl ester to phospholipid is from about 1:200 to about 1:50 w/w.

In certain embodiments, the nanoparticle includes a therapeutic agent having an X log P greater than 2.0.

Suitable therapeutic agents include analgesics/antipyretics, anesthetics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, anti-infectives, bronchodialators, hormones, hypoglycemic agents, hypolipidemic agents, proteins, nucleic acids, agents useful for erythropoiesis stimulation, antiulcer/antireflux agents, antinauseants/antiemetics, and oil-soluble vitamins. In certain embodiments, the therapeutic agent is a chemotherapeutic agent. Representative chemotherapeutic agents include taxanes, such as paclitaxel and derivatives thereof, and docetaxel and derivatives thereof. In certain embodiments, the therapeutic agent is paclitaxel.

In another aspect, the invention provides a pharmaceutical composition, comprising a nanoparticle of the invention.

In a further aspect, the invention provides a unit dosage form for treating in an individual that includes a nanoparticle of the invention and a pharmaceutically acceptable carrier.

In another aspect of the invention, a kit is provided. In one embodiment, the kit comprises a container that includes a nanoparticle of the invention, and a container comprising a pharmaceutically acceptable carrier for reconstituting the nanoparticle. In another embodiment, the kit comprises a container that includes a nanoparticle of the invention suspended in a pharmaceutically acceptable carrier. The kits optionally include instructions for using the kit in treating a disease or condition.

In a further aspect of the invention, methods of treating a disease or condition in an individual are provided. In certain embodiments, the methods comprise administering to an individual in need thereof an effective amount of the nanoparticle of the invention. In certain embodiments, the disease or condition is a proliferative disease or condition. In certain embodiments, the therapeutic agent is paclitaxel and the disease is a disease treatable by administering paclitaxel. In certain embodiments, the therapeutic agent is paclitaxel and the disease is a cancer treatable by administering paclitaxel.

In another aspect, methods for preparing a phospholipid-coated therapeutic agent nanoparticle are provided. In one embodiment, the nanoparticle is prepared by a microfluidization-solvent removal method. In another embodiment, the nanoparticle is prepared by a thin film-hydration method.

In a related aspect, the invention provides synthetic high density lipoprotein (HDL) complexes of therapeutic agents, compositions that include complexes, methods for preparing the complexes and compositions, and methods for the use of the complexes and compositions.

In one aspect, the invention provides a nanoparticle delivery vehicle comprising a high density lipoprotein complex. In one embodiment, the complex comprises:

(a) a hydrophobic core having increased hydrophobicity compared to native high density lipoprotein, the core comprising

(i) a lipid component, and

(ii) a therapeutic agent, and,

(b) a shell surrounding the core, the shell comprising a phospholipid.

In certain embodiments, the lipid component comprises apolipoprotein A1 (ApoA-1). In other embodiments, the lipid component comprises cholesterol. In further embodiments, the lipid component comprises one or more cholesterol fatty acid esters. Representative cholesterol fatty acid esters include cholesteryl laurate, cholesteryl myristate, cholesteryl palmitate, cholesteryl stearate, cholesteryl oleate, and mixtures thereof.

In certain embodiments, the lipid component comprises one or more of a sphingomyelin, a cationic phospholipid, or a glycolipid. In certain embodiments, the lipid component comprises a phosphatidylcholine. Representative phosphatidylcholines include dimyristoylphosphatidylcholine (DMPC), dioleoylphosphatidylcholine (DOPC), dipalmitoylphosphatidylcholine (DPPC), egg yolk phosphatidylcholine (egg PC), soybean phosphatidylcholine, and mixtures thereof.

In certain embodiments, the lipid component comprises a mixture of a phosphatidylcholine, cholesterol, and a cholesterol fatty acid ester.

In certain embodiments, the therapeutic agent is a poorly water-soluble therapeutic agent. Suitable therapeutic agents include chemotherapeutic agents. In one embodiment, the therapeutic agent is paclitaxel.

In certain embodiments, the phospholipid has increased hydrophobicity compared to phospholipids in native high density lipoprotein.

Suitable phospholipids include phosphatidylcholines, phosphatidylethanolamines, phosphatidylglycerols, phosphatidylserines, phosphatidylinositols, phosphatidic acids, and mixtures thereof. Representative diacylphosphatidylcholines include distearoylphosphatidylcholine (DSPC), dioleoylphosphatidylcholine (DOPC), dipalmitoylphosphatidylcholine (DPPC), dilinoleoylphosphatidylcholine DLPC), palmitoyloleoylphosphatidylcholine (POPC), palmitoyllinoleoylphosphatidylcholine, stearoyllinoleoylphosphatidylcholine stearoyloleoylphosphatidylcholine, stearoylarachidoylphosphatidylcholine, didecanoylphosphatidylcholine (DDPC), dierucoylphosphatidylcholine (DEPC), dilinoleoylphosphatidylcholine (DLOPC), dimyristoylphosphatidylcholine (DMPC), myristoylpalmitoylphosphatidylcholine (MPPC), myristoylstearoylphosphatidylcholine (MSPC), stearoylmyristoylphosphatidylcholine (SMPC), palmitoylmyristoylphosphatidylcholine (PMPC), palmitoylstearoylphosphatidylcholine (PSPC), stearoylpalmitoylphosphatidylcholine (SPPC), stearoyloleoylphosphatidylcholine (SOPC), and mixtures thereof.

In certain embodiments, the core is a substantially non-aqueous environment.

In another aspect, the invention provides a method for delivering a therapeutic agent to a subject. In one embodiment, the method comprises administering the nanoparticle delivery vehicle of the invention. In certain embodiments, the vehicle is delivered parenterally, intravenously, intramuscularly, subcutaneously, transmucosally, or transdermally.

In a further aspect, the invention provides a method for the treatment of cancer in a subject, comprising administering a therapeutically effective amount of the nanoparticle delivery vehicle of the invention to a subject in need thereof. In these methods, the cancer is treatable by the therapeutic agent. In certain embodiments, the vehicle is delivered parenterally, intravenously, intramuscularly, subcutaneously, transmucosally, or transdermally. Representative treatable cancers include prostate cancer, ovarian cancer, and breast cancer.

In another aspect, the invention provides a method for the treatment of an inflammatory disease in a subject, comprising administering a therapeutically effective amount of the nanoparticle delivery vehicle of the invention to a subject in need thereof. In these methods, the inflammatory disease is treatable by the therapeutic agent.

In a further aspect, the invention provides a method for the treatment of atherosclerosis in a subject, comprising administering a therapeutically effective amount of the nanoparticle delivery vehicle of the invention to a subject in need thereof. In these methods, atherosclerosis is treatable by the therapeutic agent.

In another aspect, the invention provides a method for making a nanoparticle delivery vehicle. In one embodiment, the method comprises:

(a) mixing lipid components in a suitable organic solvent to give a lipid mixture;

(b) adding a therapeutic agent to the lipid mixture to provide a lipid-therapeutic agent mixture;

(c) drying the lipid-therapeutic agent mixture under nitrogen to provide a solid;

(d) dispersing the solid in an aqueous solution to provide a dispersed mixture;

(e) mixing the dispersed mixture in a buffer to provide a buffered mixture;

(f) adding a suitable salt to the buffered mixture to provide a salt mixture;

(g) adding a lipid binding protein to the salt mixture to provide a lipid binding protein mixture;

(h) incubating the lipid binding protein mixture to provide an incubated mixture; and

(i) subjecting the incubated mixture to dialysis with one or more buffer changes to facilitate the self-assembly and formation of a nanoparticle delivery vehicle.

In certain embodiments, the lipid components comprise a phosphatidylcholine, cholesterol, and a cholesterol ester.

In certain embodiments, the organic solvent is dimethylsulfoxide.

In certain embodiments, the therapeutic agent is a poorly water-soluble therapeutic agent, such as a chemotherapeutic agent, for example, paclitaxel.

In a further embodiment, the invention provides a method for treating cancer in a subject by targeting cancer cells expressing a high density lipoprotein receptor. In one embodiment, the method comprises:

administering to a subject in need thereof a therapeutically effective amount of a nanoparticle delivery vehicle of the invention, whereby the therapeutic agent is transferred to an endogenous plasma high density lipoprotein to provide a high density lipoprotein particle containing the therapeutic agent;

wherein the high density lipoprotein particle containing the therapeutic agent associate with a cancer cell expressing a high density lipoprotein receptor, whereby the therapeutic agent is delivered to the cancer cell.

In certain embodiments, the high density lipoprotein receptor is a scavenger receptor type B1 (SR-B1).

In certain embodiments, administering the composition comprises systemic delivery, such as intravenous injection.

In certain embodiments, the therapeutic agent is a poorly water-soluble therapeutic agent, such as a chemotherapeutic agent, for example, paclitaxel.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a schematic illustration comparing the evolution of formulations for paclitaxel therapy.

FIG. 2 shows the chemical structures of the lipids used in the preparation of representative paclitaxel nanoparticles of the invention.

FIG. 3 compares particle size (nm) and paclitaxel entrapment efficiency (%) as a function of cholesteryl ester carbon chain length for representative paclitaxel nanoparticles of the invention.

FIG. 4 illustrates particle size and size distribution (dynamic laser light scattering) for representative paclitaxel nanoparticles of the invention (PTX-NP formulation stabilized by combination of PC-10 and cholesteryl oleate.

FIG. 5 compares particle size (nm) and paclitaxel entrapment efficiency (%) as a function of thin-film hydration water temperature for representative paclitaxel nanoparticles of the invention.

FIGS. 6A and 6B compare particle size (nm) and paclitaxel entrapment efficiency (%) as a function of amount of paclitaxel for representative paclitaxel nanoparticles of the invention prepared by thin-film hydration (Method 2) (phospholipid (PC-10) cholesteryl oleate) (LM-102) (6A), and phospholipid (PC-10)-lysophospholipid (lyso-PC-10) (LM-101) (6B).

FIGS. 7A and 7B compare nanoparticle stabilities at refrigerated temperature (4° C.) and room temperature (RT) for paclitaxel nanoparticles: representative paclitaxel nanoparticles of the invention (PC-10-cholesteryl oleate) (LM-102) (7A); and phospholipid (PC-10)-lysophospholipid (lyso-PC-10) (LM-101) (7B). The formulations were prepared by hin-film hydration (Method 2) with paclitaxel amounts of 2.5 mg to 10 mg.

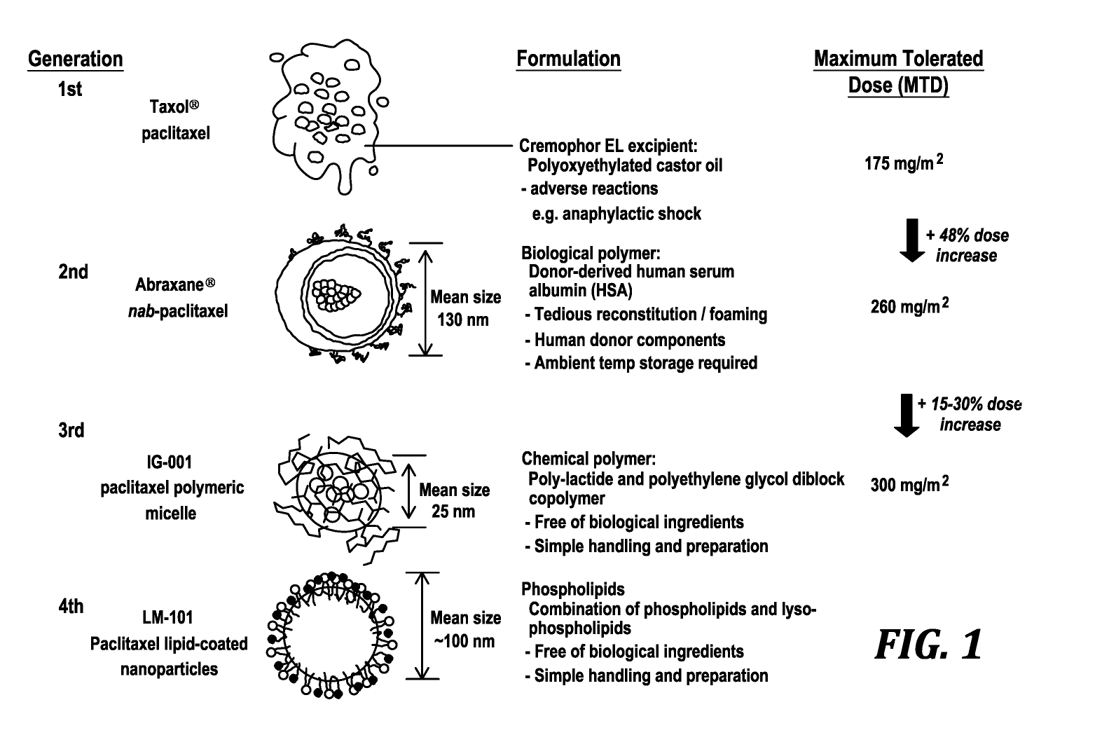

### DETAILED DESCRIPTION OF THE INVENTION

The present invention provides paclitaxel nanoparticles, paclitaxel nanoparticle formulations suitable for injection, methods for administering paclitaxel and for treating diseases and conditions treatable by paclitaxel using the formulations.

Clinically successful paclitaxel nanoparticles formulations include Abraxane® (an albumin bound nanoparticle paclitaxel) and Genexol-PM® (a polymer bound nanoparticle paclitaxel). Abraxane® contains human-derived albumin and Genexol-PM® utilizes a synthetic polymer to solubilize water insoluble paclitaxel.

The present invention utilizes biocompatible and injectable phospholipid and cholesteryl ester, components of lipoprotein nanoparticles, to provide a stable nanoparticle formulation of paclitaxel (PTX) for cancer treatment. The effect of lipid composition and methods of preparation on drug loading and physical stability of the nanoparticle formulations are described. The formulation parameters were evaluated for preparing the PTX nanoparticles included type of phospholipids and fatty acid chain lengths in cholesteryl ester, combination of phospholipid and cholesteryl esters. Process parameters, such as temperature of water for hydration, was studied and their impact on drug loading, particle size, and physical stability were evaluated. The short-term stability evaluation of nanoparticles prepared with different cholesteryl esters demonstrated that 1-10% (w/w) of cholesteryl esters produced nanoparticle with a loading of about 42% paclitaxel with particle size of less than 275 nm. The formulations were found to be stable for 24 h at 4° C. The stability of the formulations was also evaluated at different temperatures before lyophilization with different amounts of drug loading. The optimization of different parameters, such as drug amount and temperature of water for rehydration for stability of the formulations, was performed and compared to the stability of phospholipid-lysophospholipid formulations of paclitaxel (LM-101). The phospholipid-cholesteryl ester nanoparticle formulations of paclitaxel was successfully manufactured at laboratory scale.

The phospholipid-cholesteryl ester paclitaxel nanoparticle (PTX-NP) formulations were prepared by a microfluidization method (Method 1) and a thin-film evaporation (Method 2) for the preparation of phospholipid cholesteryl ester nanoformulation.

Combinative Formulation of PL, Lyso-PL, and Cholesteryl Esters by Microfluidization-Solvent Evaporation (Method 1) and Thin-Film Hydration (Method 2) Methods

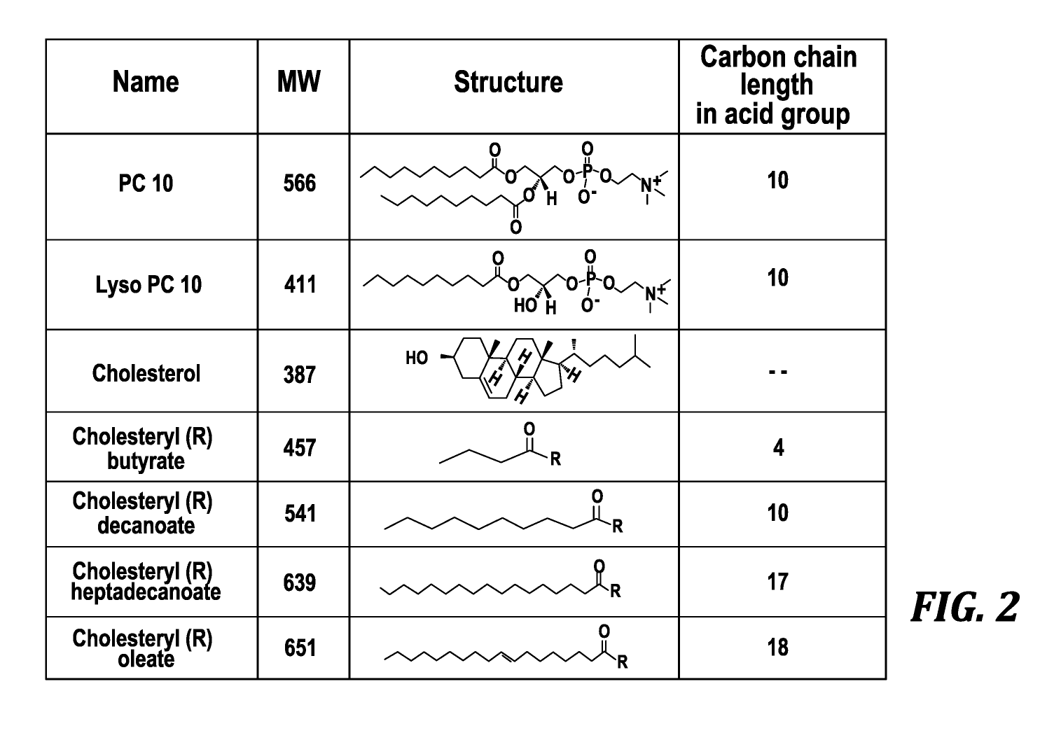

Phospholipid (PL) (PC-10-phosphatidylcholine having a C10 fatty acid component), lysophospholipid (lyso-PL) (lyso-PC-10-lysophosphatidylcholine having a C10 fatty acid component), and cholesteryl esters with different chain lengths and with significant phase transition temperature differences in the combination were used to prepare the lipid-stabilized PTX-NPs. The molecular structures of the lipids used for the studies are shown in FIG. 2. The physical properties of the PL (PC-10), lyso-PL (lyso-PC-10), cholesterol, and cholesteryl esters (butyrate, decanoate, heptadecanoate, oleate) are shown in Table 1. A series of cholesteryl esters (C4, C10, C17, and C18 fatty acid esters) was used to develop a stable lipid-based NP formulation for PTX.

TABLE 1
Physical properties of phospholipid (PL), lysophospholipid
(lyso-PL), cholesterol, and cholesteryl esters.

| Name of Lipid | MW | Transition Temp.(° C.) | CMC | |
|---|---|---|---|---|
| PC10 | 566 | ¹NA | 5 | μM |
| Lyso PC10 | 411 | NA | 6-8 | mM |
| Cholesterol | 387 | NA | 25-40 | nM |

| | | | |
|---|---|---|---|
| Cholesteryl butrate | 457 | 110 | NA |
| Cholesteryl deconate | 541 | 90 | NA |
| Cholesteryl heptadecanoate | 639 | 80 | NA |
| Cholesteryl oleate | 651 | 48 | NA |

NA—not available.

Observations:

Transition temperature of lipids decreases with increase of the carbon chain length in the fatty acid component.

Critical micelle concentration (CMC) of the lyso-PC is greater than that of the corresponding (same fatty acid carbon chain length) PC.

As shown in Table 2, the thin-film hydration method produced particles of greater paclitaxel loading than the microfluidization-solvent evaporation method. PL or lyso-PL alone did not produce particles of having a size less than about 200 nm in either method. Both methods utilizing cholesteryl esters produced particles having a size less than 200 nm.

TABLE 2
Properties of PTX-NPs prepared by thin-film hydration (Method 2) and microfluidization-solvent evaporation (Method 1)

| Method of | Phospholipid | | Lyso-phospholipid | | Lipid (cholesteryl ester) | | Paclitaxel | Particle size | Polydispersity | Entrapment |
|---|---|---|---|---|---|---|---|---|---|---|
| preparation | Name | Amt., mg | Name | Amt., mg | Name | Amt μg | (PTX), mg | (nm) | Index | Efficiency |
| Method 2 | PC10 | 40 | Lyso PC10 | 10 | Cholesteryl | 50 | 10 | 190 | 0.25 | 26 |
| | | | | | oleate | | | | | |
| | PC10 | 40 | Lyso PC10 | 10 | Cholesteryl | 500 | 10 | 172 | 0.23 | 25 |
| | | | | | oleate | | | | | |
| | PC10 | 40 | Lyso PC10 | 10 | Cholesteryl | 5000 | 10 | 159 | 0.24 | 6.4 |
| | | | | | oleate | | | | | |
| Method 1 | PC10 | 50 | Lyso PC-10 | 10 | Cholesteryl | 5000 | 10 | 156 | 0.16 | 3.2 |
| | | | | | oleate | | | | | |
| | PC10 | 50 | Lyso PC-10 | 10 | Cholesteryl | 500 | 10 | 103 | 0.14 | ND¹ |
| | | | | | oleate | | | | | |

Observations:

Particles produced by the thin-film hydration method had greater loading efficiency of drug compared to the microfludization-solvent evaporation method. Both methods produced particles having a size less than 200 nm.

Screening of Cholesteryl Ester Fatty Acid Carbon Chain Length for PTX Loading

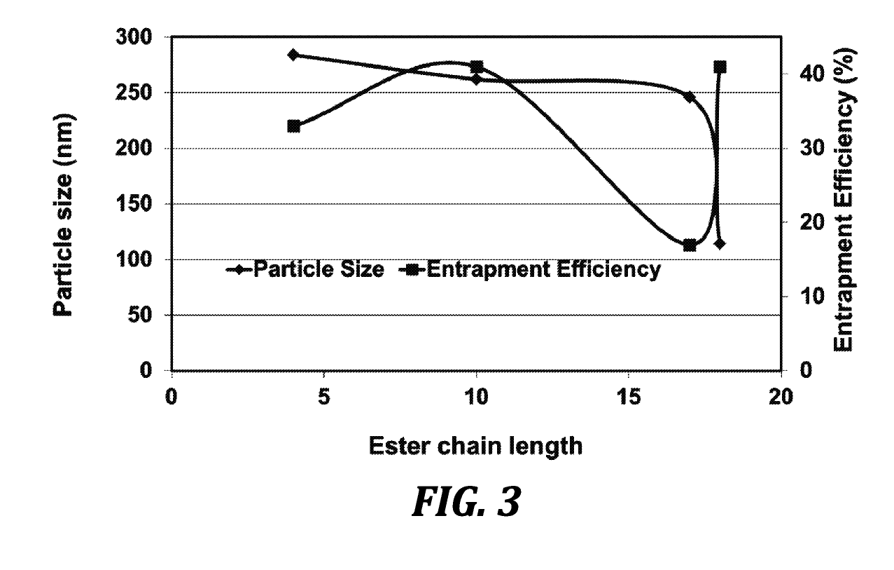

The particle size and entrapment efficiency of PTX in PC-10 and cholesteryl ester of different carbon chain length prepared by Method 2 is shown in FIG. 3. The combination of PC-10 and cholesteryl oleate produced the smallest sized particles with the greatest PTX loading.

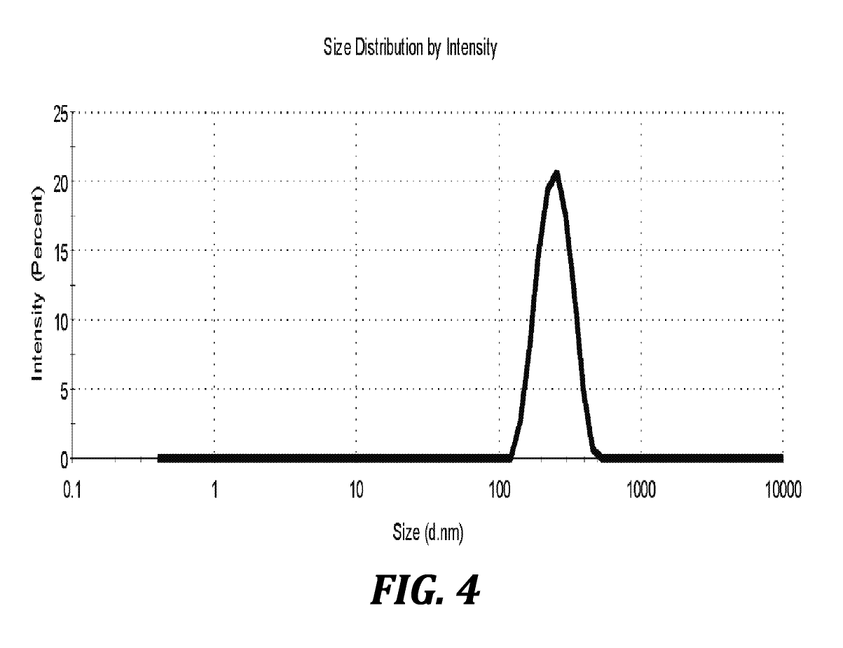

FIG. 4 illustrates particle size and size distribution (dynamic laser light scattering) for representative paclitaxel nanoparticles of the invention (PTX-NP stabilized by combination of PC-10 and cholesteryl oleate (LM-102). The cholesteryl oleate (CE) range from 0.1%, 1%, to 10% (wt/wt of total lipid weight). Optimal paclitaxel loading occurred at 1%-10% CE and 0.1% ineffective. The formulation was filterable through 0.2 μm filter and the filtered formulation has monomodal size distribution with a polydispersity index of 0.2. The Zₐᵥ of the optimized formulation was about 250 nm.

Effect of Rehydration Temperature in the Formulation Preparation Process for Paclitaxel Loading and Nanoparticles Size

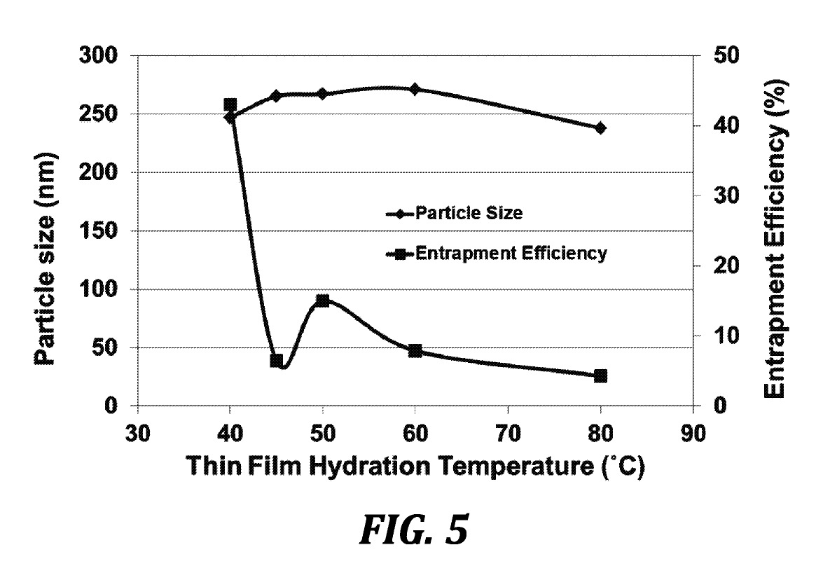

The drug loading efficiency and particle size of the formulations prepared with PC-10 and cholesteryl oleate was evaluated at water rehydration temperatures from 40° C. to 80° C. The data is shown in FIG. 5. The greatest loading of paclitaxel was obtained at 40° C. and the loading decreased as the temperature of the rehydration water increased. However, the particle size was observed to be about 250 nm for all the rehydration conditions.

Comparison of Drug Loading of PC-10-Cholesteryl Oleate (LM-102) with Phospholipid Lysophospholipid Formulation (LM-101)

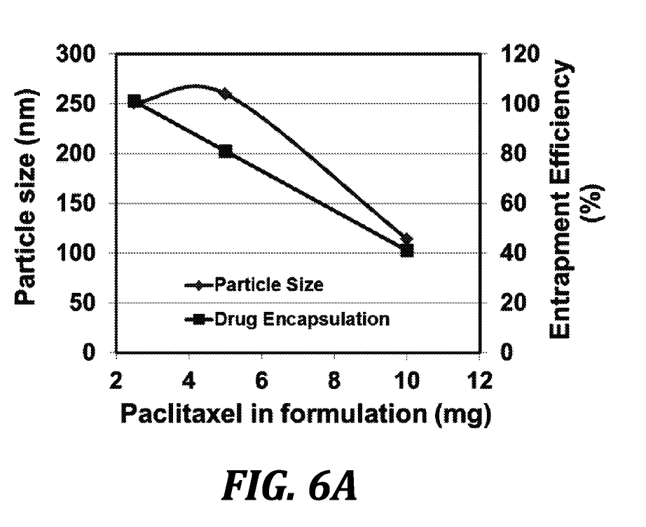

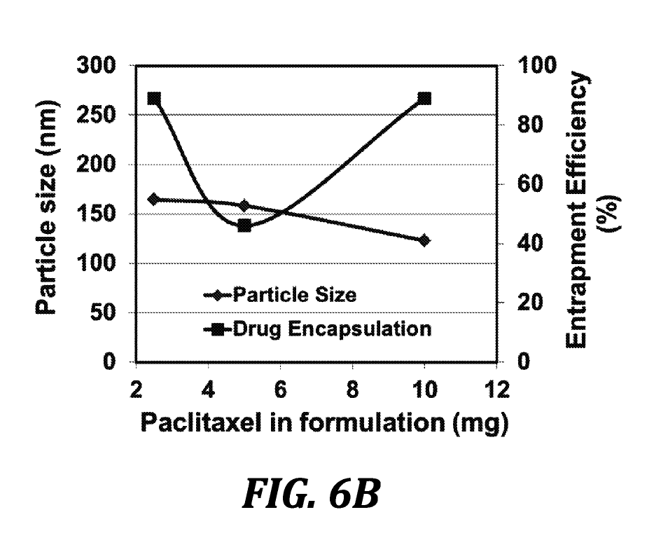

The amount of paclitaxel used in the preparation of phospholipid (PC-10) cholesteryl oleate (LM-102) (2.5 mg-10 mg paclitaxel per 50 mg of lipid) and phospholipid (PC-10)-lysophospholipid (lyso-PC-10) (80:20) affects paclitaxel loading and nanoparticle size. The effect of paclitaxel amount on particle size and entrapment efficiency for LM-102 and LM-101 prepared by Method 2 are compared in FIGS. 6A and 6B, respectively. The particle size was smaller for LM-101 for paclitaxel in the range of 2.5 mg to 10 mg. There was a significant increase in the paclitaxel entrapment efficiency for LM-102 for lower paclitaxel amounts (2.5 mg to 5.0 mg).

Comparison of Stability of PC-10-Cholesteryl Oleate (LM-102) with Phospholipid-Lysophospholipid Formulation (LM-101)

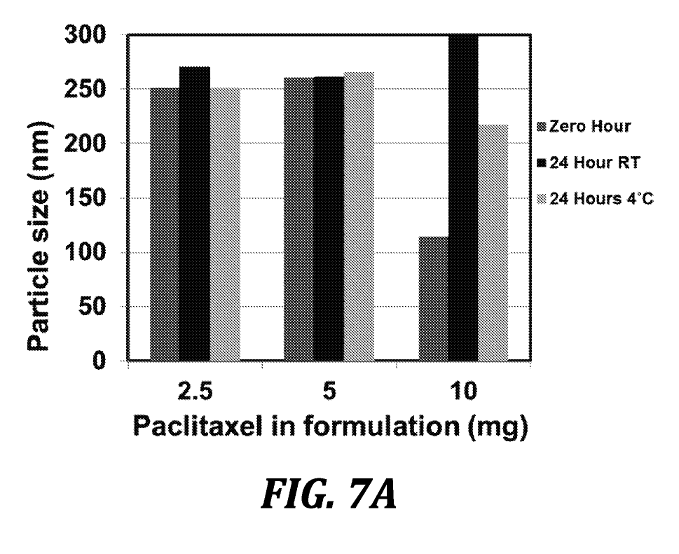

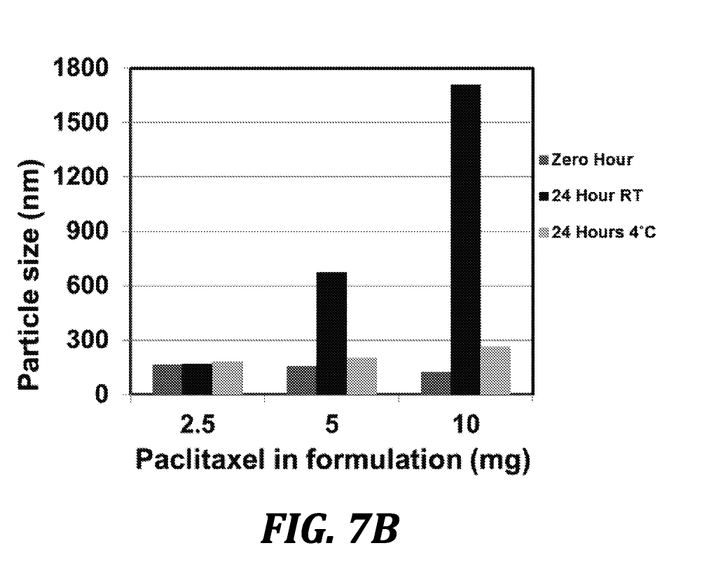

The stabilities of LM-102 and LM-101 at refrigerated temperature (4° C.) and room temperature (RT) are compared in FIGS. 7A and 7B, respectively. The formulations were prepared by thin-film hydration (Method 2) with paclitaxel amounts of 2.5 mg to 10 mg. LM-102 was found to be stable for 24 h at 4° C. for the paclitaxel loadings of 2.5 mg and 5 mg, whereas LM-101 was stable for 24 h at 4° C. for 2.5 mg drug loading.

Conclusions

The combination of PC-10 and cholesteryl oleate produced the smallest size PTX-NPs by Method 2.

The greatest entrapment efficiency (>80%) was achieved for the LM-102 formulation.

In the thin-film hydration method for nanoparticle formation, water temperature of 40° C. in the rehydration step produced the greatest paclitaxel loading.

LM-102 was stable for 24 h at 4° C.

LM-102 had a particle size of about 250 nm.

Stability of LM-102 is greater than for LM-101 for formulations with 5 mg drug loading.

### METHODS

Method 1: PTX-NPs by Microfluidization-Solvent Evaporation

Briefly, in the microfluidization-solvent evaporation method, PTX-phospholipid cholesteryl ester NPs were prepared by LV1 low volume Microfluidizer® processor microfluidization. The organic solvent containing PTX and phospholipids, and cholesteryl esters were added to an aqueous phase and the emulsion was run through the microfluidizer to obtain a nanoemulsion. The solvent from the nanoemulsion was removed by rotary evaporation to obtain nanosuspension of PTX.

Method 2: PTX-NPs by Thin-Film Hydration

Briefly, in the thin-film hydration method, the phospholipid cholesteryl ester film was prepared by dissolving PTX and phospholipids and cholesteryl ester in ethanol. The dry film was hydrated with water for visual, microscopic, size and loading efficiency measurements of the resulting unfiltered and filtered formulation

PTX-NPs Size Measurement

The particle size and the particle size distribution measurements were carried out using Zetasizer Nano-ZS and the Zₐᵥ hydrodynamic diameter of the samples was determined by cumulative analysis. The particle size and particle size distribution by intensity were measured by photon correlation spectroscopy (PCS) using dynamic laser light scattering (4 mW He—Ne laser with a fixed wavelength of 633 nm, 173° backscatter at 25° C.) in 10 mm diameter cells.

PTX Assay

An ELISA method was used to measure paclitaxel concentration in the PTX-NPs.

### Representative Embodiments

Representative phospholipid-cholesteryl ester paclitaxel nanoparticles, related formulations (e.g., LM-102), and methods for their preparation are described above. It will be appreciated that other phospholipid-cholesteryl ester paclitaxel nanoparticles and related formulations of the invention can be prepared from the components described herein, including as described below. It will also be appreciated that therapeutic agents other than paclitaxel can be formulated as phospholipid-cholesteryl ester nanoparticles as described herein, and these nanoparticles and their formulations are also within the scope of the invention.

Phospholipid-Coated Therapeutic Agent Nanoparticle

In one aspect, the invention provides phospholipid-coated therapeutic agent nanoparticle.

In one embodiment, the phospholipid-coated therapeutic agent nanoparticle, comprises a particulate therapeutic agent coated with a cholesteryl ester and one or more phospholipids, wherein the nanoparticle is stable in aqueous delivery vehicles for administration (e.g., vehicles for injection) and releases the therapeutic agent substantially instantaneously upon exposure to or contact with a physiological fluid. As used herein, the term “substantially instantaneously” refers to release of the therapeutic agent from the nanoparticle within about 1 second, within about 2 seconds, within about 5 seconds, within about 10 seconds, or within about 30 seconds after contact with a physiological fluid, such as blood, serum, plasma (e.g., intravenous injection).

In another embodiment, the phospholipid-coated therapeutic agent nanoparticle comprises a particulate therapeutic agent coated with a cholesteryl ester and one or more phospholipids, wherein the nanoparticle is as stable in aqueous delivery vehicles for administration as synthetic polymeric micelles containing a therapeutic agent (Genexol-PM®, Cynviloq®) and is as effective in releasing the therapeutic agent under physiological conditions as a human-serum albumin-coated therapeutic agent (Abraxane®).

In a further embodiment, the phospholipid-coated therapeutic agent nanoparticle comprises a particulate therapeutic agent coated with a cholesteryl ester and one or more phospholipids, wherein the phospholipid is selected from a mono-acylphospholipid, a diacylphospholipid, or a mixture thereof.

In a further embodiment, the phospholipid-coated therapeutic agent nanoparticle consists essentially of a particulate therapeutic agent coated with a cholesteryl ester and one or more phospholipids.

In another embodiment, the phospholipid-coated therapeutic agent nanoparticle consisting of a particulate therapeutic agent coated with a cholesteryl ester and one or more phospholipids.

As noted above, the invention provides a phospholipid-coated therapeutic agent nanoparticle. As used herein, “phospholipid-coated therapeutic agent nanoparticle” refers to a nanoparticle comprising a therapeutic agent in particulate form coated with a cholesteryl ester and one or more phospholipids. In the nanoparticle of the invention, the phospholipid and cholesteryl ester coating the particulate therapeutic agent advantageously stabilizes the therapeutic agent and facilitates its effective administration.

The phospholipid-coated therapeutic agent nanoparticle advantageously provides for the effective formulation and delivery of hydrophobic or substantially water insoluble therapeutic agents. Therapeutic agents advantageously formulated as nanoparticles of the invention include hydrophobic or substantially water-insoluble pharmacologically active agents (i.e., any bioactive agent having limited solubility in an aqueous or hydrophilic environment). For example, the solubility in water of these agents at 20-25° C. may be less than about 5, 2, 1, 0.5, 0.2, 0.1, 0.05, 0.02, or 0.01 mg/mL.

The character of therapeutic agents appropriate as candidate therapeutic agents that benefit from formulation as a nanoparticle of the invention include therapeutic agents, such as chemotherapeutic agents, defined by their octanol/water partition coefficient X log P (Wang et al. Chem. Inf. Comput. Sci. 1997, 37, 615-621). For example, the coefficient for paclitaxel is 3.0. In the practice of the invention, therapeutic agents with X log P greater than 2.0 are excellent candidates for incorporation into the nanoparticles of the invention. This characteristic includes over half of the approved pharmaceutical agents currently employed for parenteral administration. As used herein, the terms “hydrophobic” and “substantially water-insoluble” and “poorly water-soluble” refer to therapeutic agents having an octanol/water partition coefficient X log P greater than 2.0, and in certain embodiments greater than 3.0, and in other embodiments greater than 4.0.

Representative therapeutic agents include analgesics/antipyretics, anesthetics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, anti-infectives, bronchodialators, hormones, hypoglycemic agents, hypolipidemic agents, proteins, nucleic acids, agents useful for erythropoiesis stimulation, antiulcer/antireflux agents, antinauseants/antiemetics, and oil-soluble vitamins.

In certain embodiments, the therapeutic agent is an antineoplastic selected from adriamycin, cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, teniposide, daunomycin, indomethacin, biphenyl dimethyl dicarboxylate, interferon, camptothecin and derivatives thereof, phenesterine, paclitaxel and derivatives thereof, docetaxel and derivatives thereof, epothilones and derivatives thereof, vinblastine, vincristine, tamoxifen, etoposide, or piposulfan. Representative antineoplastic agents include taxanes and their derivatives, such as paclitaxel.

In certain embodiments, the therapeutic agent is an immunosuppressive agent selected from cyclosporine, azathioprine, mizoribine, or FK506 (tacrolimus).

The therapeutic agent nanoparticle of the invention includes one or more phospholipids coating the therapeutic agent. As used herein, the term “phospholipid” refers to a class of lipids having a hydrophobic tail (e.g., one or two) and a phosphate head group. Hydrophilicity is conferred to the phospholipid by it phosphate head group and hydrophobicity is conferred to the phospholipid by apolar groups that include long-chain saturated and unsaturated aliphatic hydrocarbon groups and such groups substituted by one or more aromatic, cycloaliphatic, or heterocyclic groups (e.g., fatty acid acyl groups).

As used herein, the term “phospholipid” refers to phosphatidic acids, phosphoglycerides, and phosphosphingolipids. Phosphatidic acids include a phosphate group coupled to a glycerol group, which may be mono- or diacylated. Phosphoglycerides (or glycerophospholipids) include a phosphate group intermediate an organic group (e.g., choline, ethanolamine, serine, inositol) and a glycerol group, which may be mono- or diacylated. Phosphosphingolipids (or sphingomyelins) include a phosphate group intermediate an organic group (e.g., choline, ethanolamine) and a sphingosine (non-acylated) or ceramide (acylated) group.

It will be appreciated that in certain embodiments, the phospholipids useful in the compositions and methods of the invention include their salts (e.g., sodium, ammonium). For phospholipids that include carbon-carbon double bonds, individual geometrical isomers (cis, trans) and mixtures of isomers are included.

Representative phospholipids include phosphatidylcholines, phosphatidylethanolamines, phosphatidylglycerols, phosphatidylserines, phosphatidylinositols, and phosphatidic acids, and their lysophosphatidyl (e.g., lysophosphatidylcholines and lysophosphatidylethanolamine) and diacyl phospholipid (e.g., diacylphosphatidylcholines, diacylphosphatidylethanolamines, diacylphosphatidylglycerols, diacylphosphatidylserines, diacylphosphatidylinositols, and diacylphosphatidic acids) counterparts.

The acyl groups of the phospholipids may be the same or different. In certain embodiments, the acyl groups are derived from fatty acids having C₁₀-C₂₄ carbon chains (e.g., acyl groups such as decanoyl (C10), dodecanoyl (also known as lauroyl) (C12), tetradecanoyl (also known as myristoyl) (C14), hexadecanoyl (also known as palmitoyl) (C16), octadecanoyl (also known as stearoyl) (C18), oleoyl, linoleoyl, linolenoyl, arachidonoyl groups).

Representative diacylphosphatidylcholines (i.e., 1,2-diacyl-sn-glycero-3-phosphocholines) include distearoylphosphatidylcholine (DSPC), dioleoylphosphatidylcholine (DOPC), dipalmitoylphosphatidylcholine (DPPC), dilinoleoylphosphatidylcholine (DLPC), palmitoyloleoylphosphatidylcholine (POPC), palmitoyllinoleoylphosphatidylcholine, stearoyllinoleoylphosphatidylcholine stearoyloleoylphosphatidylcholine, stearoylarachidoylphosphatidylcholine, didecanoylphosphatidylcholine (DDPC), didodecanoylphosphatidylcholine, dierucoylphosphatidylcholine (DEPC), dilinoleoylphosphatidylcholine (DLOPC), dimyristoylphosphatidylcholine (DMPC), myristoylpalmitoylphosphatidylcholine (MPPC), myristoylstearoylphosphatidylcholine (MSPC), stearoylmyristoylphosphatidylcholine (SMPC), palmitoylmyristoylphosphatidylcholine (PMPC), palmitoyl stearoylphosphatidylcholine (PSPC), stearoylpalmitoylphosphatidylcholine (SPPC), and stearoyloleoylphosphatidylcholine (SOPC).

Representative diacylphosphatidylethanolamines (i.e., 1,2-diacyl-sn-glycero-3-phosphoethanolamines) include dioleoylphosphatidylethanolamine (DOPE), dipalmitoylphosphatidylethanolamine (DPPE), distearoylphosphatidylethanolamine (DSPE), dilauroylphosphatidylethanolamine (DLPE), dimyristoylphosphatidylethanolamine (DMPE), dierucoylphosphatidylethanolamine (DEPE), didecanoylphosphatidylethanolamine, didodecanoylphosphatidylethanolamine, and palmitoyloleoylphosphatidylethanolamine (POPE).

Representative diacylphosphatidylglycerols (i.e., 1,2-diacyl-sn-glycero-3-phosphoglycerols) include dioleoylphosphatidylglycerol (DOPG), dipalmitoylphosphatidylglycerol (DPPG), dierucoylphosphatidylglycerol (DEPG), dilauroylphosphatidylglycerol (DLPG), dimyristoylphosphatidylglycerol (DMPG), distearoylphosphatidylglycerol (DSPG), didecanoylphosphatidylglycerol, didodecanoylphosphatidylglycerol, and palmitoyloleoylphosphatidylglycerol (POPG).

Representative diacylphosphatidylserines (i.e., 1,2-diacyl-sn-glycero-3-phosphoserines) include dilauroylphosphatidylserine (also known as didodecanoylphosphatidylserine) (DLPS), dioleoylphosphatidylserine (DOPS), dipalmitoylphosphatidylserine (DPPS), didecanoylphosphatidylserine, and distearoylphosphatidylserine (DSPS).

Representative diacylphosphatidic acids (i.e., 1,2-diacyl-sn-glycero-3-phosphates) include dierucoylphosphatidic acid (DEPA), dilauroylphosphatidic acid (also known as didodecanoylphosphatidic acid) (DLPA), dimyristoylphosphatidic acid (DMPA), dioleoylphosphatidic acid (DOPA), dipalmitoylphosphatidic acid (DPPA), didecanoylphosphatidic acid, and distearoylphosphatidic acid (DSPA).

Representative phospholipids include phosphosphingolipids such as ceramide phosphoryllipid, ceramide phosphorylcholine, and ceramide phosphorylethanolamine.

The nanoparticle of the invention includes two or more different phospholipids. In certain embodiments, the nanoparticle includes two different phospholipids. In other embodiments, the nanoparticle includes three different phospholipids. In further embodiments, the nanoparticle includes four different phospholipids.

In certain embodiments, the nanoparticle of the invention further includes a sterol (e.g., cholesterol).

As noted above, in certain embodiments, the phospholipid is a diacylphospholipid. Representative diacylphospholipids include diacylphosphatidylcholines, diacylphosphatidylethanolamines, diacylphosphatidylglycerols, diacylphosphatidylserines, diacylphosphatidylinositols, and diacylphosphatidic acids.

In certain embodiments, the diacylphospholipid (e.g., phosphatidylcholine) has a fatty acid component (acyl groups) having from 10 to 22 carbons (e.g., 10, 12, 14, 16, 18, 20, 22 carbons). In certain embodiments, the diacylphospholipid has a fatty acid component having from 10 to 20 carbons (e.g., 10, 12, 14, 16, 18, 20 carbons). In other embodiments, the diacylphospholipid has a fatty acid component having from 10 to 16 carbons (e.g., 10, 12, 14, 16 carbons). In further embodiments, the diacylphospholipid has a fatty acid component having from 10 to 14 carbons (e.g., 10, 12, 14 carbons). In yet other embodiments, the diacylphospholipid has a fatty acid component having from 10 to 12 carbons (e.g., 10, 12 carbons). In certain embodiments, the diacylphospholipid has a fatty acid component having 10 carbons. It will be appreciated that in certain of the embodiments noted above, each of the fatty acid components in the diacylphospholipid has the same number of carbons (e.g., 10, 12, 14, 16, 18, 20, 22 carbons), such as 1,2-didecanoylphosphatidylcholine, and that in other of the embodiments noted above, each of the fatty acid components in the diacylphospholipid has a different number of carbons, such as stearoyloleoylphosphatidylcholine.

In certain embodiments, the phospholipid is a phosphatidylcholine. Suitable phosphatidylcholines include phosphatidylcholines having a fatty acid component (acyl groups) having from 10 to 22 carbons (e.g., 10, 12, 14, 16, 18, 20, 22 carbons). In certain embodiments, the phosphatidylcholine has a fatty acid component having from 10 to 20 carbons (e.g., 10, 14, 16, 18, 20 carbons). In other embodiments, the phosphatidylcholine has a fatty acid component having from 10 to 16 carbons (e.g., 10, 12, 14, 16 carbons). In further embodiments, the phosphatidylcholine has a fatty acid component having from 10 to 14 carbons (e.g., 10, 12, 14 carbons). In yet other embodiments, the phosphatidylcholine has a fatty acid component having from 10 to 12 carbons (e.g., 10, 12 carbons). In certain embodiments, the phosphatidylcholine has a fatty acid component having 10 carbons. It will be appreciated that the fatty acid component of a particular phospholipid need not be the same (i.e., diacyl with different acyl groups).

In certain embodiments, the phospholipid is an electronically neutral phospholipid having, for example, a negatively charged phosphate group and a positively charged amine group (e.g., a phosphatidylcholine or phosphatidylethanolamine). In other embodiments, the phospholipid is an electronically negative phospholipid having a negatively charged phosphate group (e.g., a phosphatidylglycerol).

As noted above, in certain embodiments, the phospholipid is a lysophospholipid. In certain of these embodiments, the lysophospholipid is a mono-acylphospholipid. Representative lysophospholipids include lysophosphatidylcholines, lysophosphatidylethanolamines, lysophosphatidylglycerols, lysophosphatidylserines, lysophosphatidylinositols, and lysophosphatidic acids (e.g., mono-acylphosphatidyl compounds).

In certain embodiments, the mono-acylphospholipid (e.g., lysophosphatidylcholine) has a fatty acid component (acyl group) having from 10 to 22 carbons (e.g., 10, 12, 14, 16, 18, 20, 22 carbons). In certain embodiments, the mono-acylphospholipid has a fatty acid component having from 10 to 20 carbons (e.g., 10, 12, 14, 16, 18, 20 carbons). In other embodiments, the mono-acylphospholipid has a fatty acid component having from 10 to 16 carbons (e.g., 10, 12, 14, 16 carbons). In further embodiments, the mono-acylphospholipid has a fatty acid component having from 10 to 14 carbons (e.g., 10, 12, 14 carbons). In yet other embodiments, the mono-acylphospholipid has a fatty acid component having from 10 to 12 carbons (e.g., 10, 12 carbons). In certain embodiments, the mono-acylphospholipid has a fatty acid component having 10 carbons.

In certain embodiments, the phospholipid is a lysophosphatidylcholine. Suitable lysophosphatidylcholines include lysophosphatidylcholines having a fatty acid component (acyl group) having from 10 to 22 carbons (e.g., 10, 12, 14, 16, 18, 20, 22 carbons). In certain embodiments, the phosphatidylcholine has a fatty acid component having from 10 to 20 carbons (e.g., 10, 14, 16, 18, 20 carbons). In other embodiments, the lysophosphatidylcholine has a fatty acid component having from 10 to 16 carbons (e.g., 10, 12, 14, 16 carbons). In further embodiments, the lysophosphatidylcholine has a fatty acid component having from 10 to 14 carbons (e.g., 10, 12, 14 carbons). In yet other embodiments, the lysophosphatidylcholine has a fatty acid component having from 10 to 12 carbons (e.g., 10, 12 carbons). In certain embodiments, the lysophosphatidylcholine has a fatty acid component having 10 carbons.

In certain embodiments, the lysophospholipid is an electronically neutral lysophospholipid having, for example, a negatively charged phosphate group and a positively charged amine group (e.g., a lysophosphatidylcholine or lysophosphatidylethanolamine). In other embodiments, the lysophospholipid is an electronically negative lysophospholipid having a negatively charged phosphate group (e.g., a lysophosphatidylglycerol).

In certain embodiments, the nanoparticle of the invention includes a diacylphospholipid and a mono-acylphospholipid. In certain of these embodiments, the nanoparticle of the invention includes a phosphatidylcholine and a lysophosphatidylcholine.

The ratio of di- to mono-acylphospholipid (e.g., phosphatidylcholine to lysophosphatidylcholine) is from about 1:99 w/w percent to about 99:1 w/w percent. In certain embodiments, the ratio of di- to mono-acylphospholipid (e.g., phosphatidylcholine to lysophosphatidylcholine) is about 10:90 to about 90:10 w/w percent. In other embodiments, the ratio of di- to mono-acylphospholipid (e.g., phosphatidylcholine to lysophosphatidylcholine) is about 20:80 to about 80:20 w/w percent. In further embodiments, the ratio of di- to mono-acylphospholipid (e.g., phosphatidylcholine to lysophosphatidylcholine) is about 30:70 to about 70:30 w/w percent. In other embodiments, the ratio of di- to mono-acylphospholipid (e.g., phosphatidylcholine to lysophosphatidylcholine) is about 40:60 to about 60:40 w/w percent. In certain embodiments, the ratio of di- to mono-acylphospholipid (e.g., phosphatidylcholine to lysophosphatidylcholine) is about 50:50 w/w percent. In certain embodiments, the ratio of di- to mono-acylphospholipid (e.g., phosphatidylcholine to lysophosphatidylcholine) is from about 90:10 to about 60:40 w/w percent. In other embodiments, the ratio of di- to mono-acylphospholipid (e.g., phosphatidylcholine to lysophosphatidylcholine) is from about 90:10 to about 70:30 w/w percent. In further embodiments, the ratio of di- to mono-acylphospholipid (e.g., phosphatidylcholine to lysophosphatidylcholine) is about 80:20 w/w percent.

In certain embodiments of the invention in which the nanoparticle includes a diacylphospholipid (e.g., a phosphatidylcholine) and a mono-acylphospholipid (e.g., lysophosphatidylcholine), the fatty acid components of the di- and mono-acylphospholipids are the same. For example, each of the di- and mono-acylphospholipids includes C10 (decanoyl) fatty acid components, each includes C12 (dodecanoyl) fatty acid components, each includes C14 (tetradecanoyl) fatty acid components, each includes C16 (hexadecanoyl) fatty acid components, or each includes C18 (dodecanoyl) fatty acid components. Alternatively, in other embodiments, the fatty acid components of the di- and mono-acylphospholipids are different (e.g., the diacylphospholipid includes C10 fatty acid components and the mono-acylphospholipid includes a C12 fatty acid component.

In addition to the phospholipid, the therapeutic agent nanoparticle of the invention includes one or more cholesteryl esters. As used herein, the term “cholesteryl ester” refers to an ester of cholesterol that may be produced from esterification of cholesterol with a carboxylic acid. The ester bond is formed between the carboxylate group of the carboxylic acid (e.g., fatty acid) and the hydroxyl group of cholesterol. In certain embodiments, the cholesteryl ester is a cholesteryl fatty acid ester (i.e., the carboxylic acid in the production process is a fatty acid). Cholesteryl esters can be characterized by the nature of carboxylic acid component. Representative cholesteryl esters include short-chain carboxylic acid components having less than six carbons, medium-chain carboxylic acid components having from six to twelve carbons, long-chain carboxylic acid components having from thirteen to twenty-one carbons, and very long-chain carboxylic acid components having greater than twenty-two carbons.

The carboxylic acid components may have carbon chains that are straight chains or branched chains. The carboxylic acid components may have carbon chains that saturated or unsaturated (having one or more carbon-carbon double bonds). The configuration of each double bond may be cis or trans.

In certain embodiments, the carboxylic acid component of the cholesteryl ester has from 4 to 18 carbons. In other embodiments, the carboxylic acid component of the cholesteryl ester has from 8 to 16 carbons. In further embodiments, the carboxylic acid component of the cholesteryl ester has from 8 to 12 carbons. In one embodiment, the carboxylic acid component of the cholesteryl ester has 10 carbons. In another embodiment, the carboxylic acid component of the cholesteryl ester has 12 carbons. In a further embodiment, the carboxylic acid component of the cholesteryl ester has 18 carbons.

In certain embodiments, the cholesteryl ester is present in the nanoparticle in an amount from about 0.1 to about 10% by weight of the total amount of phospholipid(s). In other embodiments, the cholesteryl ester is present in the nanoparticle in an amount from about 1 to about 10% by weight of the total amount of phospholipid(s). In further embodiments, the cholesteryl ester is present in the nanoparticle in an amount from about 5 to about 10% by weight of the total amount of phospholipid(s).

The cholesteryl ester, phospholipid, and therapeutic agent in the composition can be associated in various manners. For example, in some embodiments, the cholesteryl ester and phospholipid is an admixture with the therapeutic agent. In some embodiments, the cholesteryl ester and phospholipid encapsulate or entrap the therapeutic agent. In some embodiments, the cholesteryl ester and phospholipid are bound (e.g., non-covalently bound) to the therapeutic agent.

Particle Size.

The nanoparticles of the invention have an average or mean diameter of no greater than about any of about 1000, 900, 800, 700, 600, 500, 400, 300, 200, or 100 nm. In some embodiments, the average or mean diameter of the particles is between about 30-300 nm. In some embodiments, the average or mean diameter of the particles is between about 20-200 nm. In some embodiments, the average or mean diameter of the particles is between about 80-200 nm. In certain embodiments, the average or mean diameter of the particles is between about 30-180 nm. In some embodiments, the average or mean diameter of the particles is between about 40-160 nm. In certain embodiments, the average or mean diameter of the particles is between about 80-140 nm. In other embodiments, the average or mean diameter of the particles is between about 90-120 nm. In some embodiments, the particles are sterile-filterable.

Particle Charge.

Depending on the composition of the phospholipids of the nanoparticle, the nanoparticle can be electronically neutral or charged. In certain embodiments, when the nanoparticle includes only phospholipids (e.g., di- and/or mono-acylphospholipids) that are electronically neutral (e.g., a phosphatidylcholine, lysophosphatidylcholine, phosphatidylethanolamine, and/or lyso phosphatidylethanolamine, each having a negatively charged phosphate group and a positively charged amine group), the nanoparticle is electronically neutral, at least in regard to the nanoparticle's phospholipid component. In other embodiments, when the nanoparticle includes a phospholipid (e.g., di- and/or mono-acylphospholipid) that is negatively charged (e.g., a phosphatidylglycerol having a negatively charged phosphate group and no corresponding positively charged group), the nanoparticle is electronically negative, at least in regard to the nanoparticle's phospholipid component.

In certain embodiments, the nanoparticle is electronically neutral in regard to the nanoparticle's phospholipid content. In other embodiments, the nanoparticle is electronically negative (negatively charged) in regard to the nanoparticle's phospholipid content.

Representative nanoparticles of the invention demonstrate pharmacokinetic bioequivalence to Abraxane® with large volume of distribution, low AUC, and low Cmax in comparison to solvent-based paclitaxel formulations, such as Taxol® or Tocosol®. Cynviloq® provides the desired pharmacokinetic bioequivalence to Abraxane®, but suffers from undesirable hypersensitivity to its excipient/polymer. This prompted the replacement of mPEG-PDLLA in Cynviloq® with naturally-occurring phospholipids.

It will be appreciated that in certain embodiments, the nanoparticles of the invention comprise the components described herein. In certain other embodiments, it will be appreciated that the nanoparticles of the invention consist essentially of the components described herein, and that in these embodiments the nanoparticles do not include any additional component that would material affect the properties of the nanoparticle (e.g., therapeutic function, effect, or other pharmacokinetic properties). In certain further embodiments, it will be appreciated that the nanoparticles of the invention consist of the components described herein, and that in these embodiments the nanoparticles do not include any additional components.

Phospholipid-Coated Therapeutic Agent Nanoparticle Compositions

In another aspect of the invention, phospholipid-coated therapeutic agent nanoparticle compositions are provided. Representative compositions include dry and liquid compositions.

In certain embodiments, the composition comprises a dry (e.g., lyophilized) composition. In other embodiments, the composition is a liquid (e.g., aqueous) composition obtained by reconstituting or resuspending a dry composition. In further embodiments, the composition is an intermediate liquid (e.g., aqueous) composition that can be dried (e.g., lyophilized).

Dry compositions of the invention can be reconstituted, resuspended, or rehydrated to form generally a stable aqueous suspension of particles comprising the therapeutic agent and phospholipid (e.g., phospholipid-coated therapeutic agent). A hydrophobic therapeutic agent is “stabilized” by a phospholipid in an aqueous suspension if it remains suspended in an aqueous medium (e.g., without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to a subject (e.g., human). The stability of the suspension is in some embodiments evaluated at room temperature (e.g., 20-25° C.) or refrigerated conditions (e.g., 4° C.). Stability can also be evaluated under accelerated testing conditions, such as at a temperature that is higher than about 40° C.

In other embodiments, the composition is a liquid (e.g., aqueous) composition obtained by reconstituting or resuspending a dry composition in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

The amount of phospholipid in the composition described herein will vary depending on the therapeutic agent and other components in the composition. In some embodiments, the composition comprises a phospholipid in an amount that is sufficient to stabilize the therapeutic carrier in an aqueous suspension, for example, in the form of a stable colloidal suspension (e.g., a stable suspension of nanoparticles). In some embodiments, the phospholipid is in an amount that reduces the sedimentation rate of the therapeutic agent in an aqueous medium. For particle-containing compositions, the amount of the phospholipid also depends on the size and density of particles of the therapeutic agent.

In some embodiments, the phospholipid is present in an amount that is sufficient to stabilize the therapeutic agent in an aqueous suspension at a certain concentration. For example, the concentration of the therapeutic agent in the composition is about 0.1 to about 100 mg/ml, including, for example, any of about 0.1 to about 50 mg/ml, about 0.1 to about 20 mg/ml, about 1 to about 10 mg/ml, about 2 to about 8 mg/ml, and about 4 to about 6 mg/ml. In some embodiments, the concentration of the therapeutic agent is at least about any of about 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the phospholipid is present in an amount that avoids use of surfactants (such as Tween 80 or Cremophor or other biocompatible polymers). Thus, in certain embodiments, the compositions of the invention are advantageously free or substantially free of surfactants (such as Tween 80 and Cremophor) and other biocompatible polymers (e.g., serum albumins, such as human serum albumin, and synthetic polymers such as poly(alkylene oxide)-containing polymers as described in U.S. Pat. No. 6,322,805).

In some embodiments, the composition, in liquid form, comprises from about 0.1% to about 50% (w/v) (e.g., about 0.5% (w/v), about 5% (w/v), about 10% (w/v), about 15% (w/v), about 20% (w/v), about 30% (w/v), about 40% (w/v), about 50% (w/v)) of the phospholipid. In some embodiments, the composition, in liquid form, comprises about 0.5% to about 5% (w/v) of the phospholipid.

In some embodiments, the weight ratio of phospholipid to the therapeutic agent is such that a sufficient amount of the therapeutic agent binds to, or is transported by, the cell. While the weight ratio of phospholipid to therapeutic agent can be optimized for different phospholipid and therapeutic agent combinations, generally the weight ratio of phospholipid to therapeutic agent (w/w) is about 0.01:1 to about 100:1, including for example any of about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, and about 9:1. In some embodiments, the phospholipid to therapeutic agent weight ratio is about any of 18:1 or less, such as about any of 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less.

In some embodiments, the phospholipid allows the composition to be administered to a subject (e.g., human) without significant side effects. In some embodiments, the phospholipid is in an amount that is effective to reduce one or more side effects of administration of the therapeutic agent to a human. The term “reducing one or more side effects of administration of the therapeutic agent” refers to reduction, alleviation, elimination, or avoidance of one or more undesirable effects caused by the therapeutic agent, as well as side effects caused by delivery vehicles (such as solvents that render the therapeutic suitable for injection) used to deliver the therapeutic agent. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with various therapeutic agents can be reduced.

Other Components in the Compositions.

The compositions described herein can include other agents, excipients, or stabilizers to improve properties of the composition. Examples of suitable excipients and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate and mineral oil. The formulations can additionally include lubricating agents, wetting agents, emulsifying and suspending agents, preserving agents, sweetening agents or flavoring agents. Examples of emulsifying agents include tocopherol esters such as tocopheryl polyethylene glycol succinate and the like, Pluronic, emulsifiers based on polyoxyethylene compounds, Span 80 and related compounds, and other emulsifiers known in the art and approved for use in animals or human dosage forms. The compositions can be formulated so as to provide rapid, sustained or delayed release of the active ingredient after administration to the patient by employing procedures well known in the art.

Compositions for administration by injection include those comprising a therapeutic agent as the active ingredient in association with a surface-active agent (or wetting agent or surfactant), or in the form of an emulsion (e.g., as a water-in-oil or oil-in-water emulsion). Other ingredients can be added, for example, mannitol or other pharmaceutically acceptable vehicles, if necessary.

In some embodiments, the composition is suitable for administration to a human. In some embodiments, the composition is suitable for administration to a mammal, such as, in the veterinary context, including domestic pets and agricultural animals. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, (d) suitable emulsions, and (e) powders. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH in the range of about 4.5 to about 9.0, including for example pH in the ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 7.5 or about 8). The composition can also be made to be isotonic with blood by the addition of a suitable tonicity modifier, such as glycerol.

It will be appreciated that in certain embodiments, the compositions of the invention comprise the components described herein (e.g., may include other component such as described below). In certain other embodiments, it will be appreciated that the compositions of the invention consist essentially of the components described herein, and that in these embodiments the compositions do not include any additional component that would material affect the properties of the nanoparticle (e.g., therapeutic function, effect, or other pharmacokinetic properties). In certain further embodiments, it will be appreciated that the compositions of the invention consist of the components described herein, and that in these embodiments the compositions do not include any additional components.

Articles of Manufacture Comprising Phospholipid-Coated Therapeutic Agent Nanoparticles

In further aspects, the invention provides articles of manufacture comprising the compositions described herein in suitable packaging. Suitable packaging for compositions described herein are known in the art, and include, for example, vials (such as sealed vials), vessels (such as sealed vessels), ampules, bottles, jars, flexible packaging (such as sealed Mylar or plastic bags), and the like. These articles of manufacture may further be sterilized and/or sealed.

Also provided are unit dosage forms comprising the compositions described herein. These unit dosage forms can be stored in a suitable packaging in single or multiple unit dosages and may also be further sterilized and sealed.

The present invention also provides kits comprising compositions (or unit dosages forms and/or articles of manufacture) described herein and may further comprise instruction(s) on methods of using the composition, such as uses further described herein. In some embodiments, the kit of the invention comprises the packaging described above. In other embodiments, the kit of the invention comprises the packaging described above and a second packaging comprising a buffer. It may further include other materials desirable from a commercial and user standpoint, including other buffers, diluents, filters, needles, syringes, and package inserts with instructions for performing any methods described herein.

Kits may also be provided that contain sufficient dosages of the therapeutic agent as disclosed herein to provide effective treatment for an individual for an extended period, such as any of a week, 2 weeks, 3 weeks, 4 weeks, 6 weeks, 8 weeks, 3 months, 4 months, 5 months, 6 months, 7 months, 8 months, 9 months or more. Kits may also include multiple unit doses of the therapeutic agent and pharmaceutical compositions and instructions for use and packaged in quantities sufficient for storage and use in pharmacies, for example, hospital pharmacies and compounding pharmacies.

Methods of Using Phospholipid-Coated Therapeutic Agent Nanoparticles

In another aspect, the invention provides methods for using the phospholipid-coated therapeutic agent nanoparticles.

In certain embodiments, the invention provides a method for treating a disease or condition that is responsive to a therapeutic agent comprising administering a composition comprising an effective amount of the phospholipid-coated therapeutic agent nanoparticle. For example, in some embodiments, there is provided a method of treating cancer in an individual (such as human) comprising administering to the individual a composition comprising an effective amount of a antineoplastic therapeutic agent (such as paclitaxel) and a phospholipid protein.

The term “effective amount” used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth). In some embodiments, an effective amount is an amount sufficient to delay development. In some embodiments, an effective amount is an amount sufficient to prevent occurrence and/or recurrence. An effective amount can be administered in one or more administrations.

The compositions of the invention (e.g., where the therapeutic agent is an anti-proliferative agent, such as paclitaxel) are effective for treating proliferative diseases including cancers, restenosis, and fibrosis, among others. When the therapeutic agent is paclitaxel, the compositions are effective for treating diseases and conditions treatable by administering paclitaxel.

Cancers to be treated by compositions described herein include, but are not limited to, carcinoma, lymphoma, blastoma, sarcoma, and leukemia. Examples of cancers that can be treated by compositions described herein include, but are not limited to, squamous cell cancer, lung cancer (including small cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, melanoma, endometrical or uterine carcinoma, salivary gland carcinoma, kidney or renal cancer, liver cancer, prostate cancer, vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome. In some embodiments, there is provided a method of treating metastatic cancer (that is, cancer that has metastasized from the primary tumor). In some embodiments, there is provided a method of reducing cell proliferation and/or cell migration. In some embodiments, there is provided a method of treating hyperplasia.

In some embodiments, there are provided methods of treating cancer at advanced stage(s). In some embodiments, there are provided methods of treating breast cancer (which may be HER2 positive or HER2 negative), including, for example, advanced breast cancer, stage IV breast cancer, locally advanced breast cancer, and metastatic breast cancer. In some embodiments, the cancer is lung cancer, including, for example, non-small cell lung cancer (NSCLC, such as advanced NSCLC), small cell lung cancer (SCLC, such as advanced SCLC), and advanced solid tumor malignancy in the lung. In some embodiments, the cancer is ovarian cancer, head and neck cancer, gastric malignancies, melanoma (including metastatic melanoma), colorectal cancer, pancreatic cancer, and solid tumors (such as advanced solid tumors). In some embodiments, the cancer is any of (and in some embodiments selected from the group consisting of) breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, gliomas, glioblastomas, neuroblastomas, and multiple myeloma. In some embodiments, the cancer is a solid tumor. In some embodiments, the cancer is any of (in some embodiments, selected from the group consisting of) prostate cancer, colon cancer, breast cancer, head and neck cancer, pancreatic cancer, lung cancer, and ovarian cancer.

Individuals suitable for receiving these compositions depend on the nature of the therapeutic agent, as well as the disease/condition/disorder to be treated and/or prevented. Accordingly, the terms “individual” and “subject” include any of vertebrates, mammals, and humans depending on intended suitable use. In some embodiments, the individual is a mammal. In some embodiments, the individual is any one or more of human, bovine, equine, feline, canine, rodent, or primate. In some embodiments, the individual is a human.

In further embodiments, the invention provides a method of treating carcinoma (such as colon carcinoma) in an individual, wherein the method comprises administering to the individual a composition comprising an effective amount of phospholipid-coated therapeutic agent nanoparticle.

The compositions described herein can be administered alone or in combination with other pharmaceutical agents, including poorly water soluble pharmaceutical agents. For example, when the composition comprises a taxane (such as paclitaxel), it can be co-administered with one or more other chemotherapeutic agents including, but are not limited to, carboplatin, Navelbine (vinorelbine), anthracycline (Doxil), lapatinib (GW57016), Herceptin, gemcitabine (Gemzar), capecitabine (Xeloda), alimta, cisplatin, 5-fluorouracil, epirubicin, cyclophosphamide, avastin, Velcade. In some embodiments, the taxane composition is co-administered with a chemotherapeutic agent selected from the group consisting of antimetabolites (including nucleoside analogs), platinum-based agents, alkylating agents, tyrosine kinase inhibitors, anthracycline antibiotics, vinca alkloids, proteasome inhibitors, macrolides, and topoisomerase inhibitors. These other pharmaceutical agents can be present in the same composition as the drug (such as taxane), or in a separate composition that is administered simultaneously or sequentially with the drug (such as taxane)-containing composition.

The dose of the composition of the invention administered to an individual will vary with the particular composition, the method of administration, and the particular disease being treated. The dose is sufficient to effect a desirable response, such as a therapeutic or prophylactic response against a particular disease or condition. For example, the dosage of representative therapeutic agents (e.g., paclitaxel) administered can be about 1 to about 300 mg/m², including for example about 10 to about 300 mg/m², about 30 to about 200 mg/m², and about 70 to about 150 mg/m². Typically, the dosage of a therapeutic agent (e.g., paclitaxel) in the composition can be in the range of about 50 to about 200 mg/m² when given on a 3 week schedule, or about 10 to about 100 mg/m² when given on a weekly schedule. In addition, if given in a metronomic regimen (e.g., daily or a few times per week), the dosage may be in the range of about 1-50 mg/m².

Dosing frequency for the compositions of the invention includes, but is not limited to, at least about any of once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequent.

The administration of the compositions of the invention can be extended over an extended period of time, such as from about a month up to about three years. For example, the dosing can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The compositions described herein can be administered to an individual via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In certain embodiments, the compositions are administered by any acceptable route including, but not limited to, orally, intramuscularly, transdermally, and intravenously.

When preparing the compositions for injection, particularly for intravenous delivery, the continuous phase preferably comprises an aqueous solution of tonicity modifiers, buffered to a pH range of about 5 to about 8.5. The pH may also be below 7 or below 6. In some embodiments, the pH of the composition is no less than about 6, including for example no less than about any of 6.5, 7, or 8 (such as about 7.5 or 8).

The nanoparticles of this invention can be enclosed in a hard or soft capsule, can be compressed into tablets, or can be incorporated with beverages or food or otherwise incorporated into the diet. Capsules can be formulated by mixing the nanoparticles with an inert pharmaceutical diluent and inserting the mixture into a hard gelatin capsule of the appropriate size. If soft capsules are desired, a slurry of the nanoparticles with an acceptable vegetable oil, light petroleum or other inert oil can be encapsulated by machine into a gelatin capsule.

In the practice of the methods of use, the invention provides methods of reducing side effects associated with administration of a therapeutic agent to a human, comprising administering to a human a pharmaceutical composition comprising the phospholipid-coated therapeutic agent nanoparticle. For example, the invention provides methods of reducing various side effects associated with administration of the therapeutic agent, including, but not limited to, myelosuppression, neurotoxicity, hypersensitivity, inflammation, venous irritation, phlebitis, pain, skin irritation, peripheral neuropathy, neutropenic fever, anaphylactic reaction, hematologic toxicity, and cerebral or neurologic toxicity, and combinations thereof. In some embodiments, there is provided a method of reducing hypersensitivity reactions associated with administration of the therapeutic agent, including, for example, severe skin rashes, hives, flushing, dyspnea, tachycardia, and others.

Methods of Making Phospholipid-Coated Therapeutic Agent Nanoparticles

In another aspect, the invention provides methods for making the phospholipid-coated therapeutic agent nanoparticles.

In certain embodiments, the methods for the formation of nanoparticles of the invention include preparation under conditions of high shear forces (e.g., sonication, high pressure homogenization, or the like). Representative methods for forming nanoparticles under high shear force conditions are described in U.S. Pat. Nos. 5,916,596; 6,506,405; and 6,537,579, incorporated herein by reference.

Briefly, in certain embodiments, the therapeutic agent is dissolved in an organic solvent to provide a solution that is combined with an aqueous phospholipid solution to provide a mixture. The mixture is subjected to high pressure homogenization. Post-homogenization to the desired level, the organic solvent is removed by evaporation to provide an aqueous dispersion. The dispersion obtained can be further lyophilized to provide a particulate solid.

Suitable organic solvents include solvents in which the therapeutic agent is soluble, that are miscible with aqueous solution, and that can be removed by evaporation at reasonable temperature and pressure. Representative useful solvents include ketones, esters, ethers, chlorinated solvents, and other solvents known in the art. For example, the organic solvent can be methylene chloride or chloroform/ethanol (for example, with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1).

Pharmaceutically acceptable excipients can also be added to the composition. Suitable pharmaceutically acceptable excipients include solutions, emulsions, or suspensions.

Other emulsion or nanoparticle formulations may also be prepared. An emulsion is formed by homogenization under high pressure and high shear forces. Such homogenization is conveniently carried out in a high-pressure homogenizer, typically operated at pressures in the range of about 3,000 up to 30,000 psi. Preferably, such processes are carried out at pressures in the range of about 6,000 up to 25,000 psi. The resulting emulsion comprises very small nanodroplets of the non-aqueous solvent containing the dissolved therapeutic agent and very small nanodroplets of the phospholipid. Acceptable methods of homogenization include processes imparting high shear and cavitation such as, for example, high-pressure homogenization, high shear mixers, sonication, high shear impellers and the like.

Colloidal systems prepared in accordance with the present invention can be further converted into powder form by removal of the water (e.g., lyophilization) at a suitable temperature-time profile. The lyophilized product (e.g., particulate powder) is readily reconstituted by addition of water, saline or buffer, without the need to use conventional cryoprotectants such as mannitol, sucrose, glycine and the like. While not required, it is of course understood that conventional cryoprotectants can be added to the pharmaceutical compositions if so desired.

In one embodiment, the nanoparticles are prepared by microfluidization-solvent evaporation.

In another embodiment, the nanoparticles are prepared by thin-film hydration. Briefly, in this method, phospholipids and paclitaxel were dissolved in ethanol and subjected to rotary evaporation until a thin film was formed and all the solvents were evaporated. The film was then hydrated using deionized (DI) water to produce paclitaxel-loaded phospholipid nanoparticles.

The methods of the invention include methods of making pharmaceutical compositions comprising combining any of the compositions described herein with a pharmaceutically acceptable excipient.

In a further aspect, the invention provides use of the compositions described herein in the manufacture of a medicament. Particularly, the manufacture of a medicament for use in the treatment of conditions described herein. Further, the pharmaceutical composition thereof described herein, are also intended for use in the manufacture of a medicament for use in treatment of the conditions and, in accordance with the methods, described herein.

Synthetic High Density Lipoprotein (HDL) Complexes of Therapeutic Agents

In a related aspect, the invention provides synthetic high density lipoprotein (HDL) complexes of therapeutic agents, compositions that include complexes, methods for preparing the complexes and compositions, and methods for the use of the complexes and compositions.

In one aspect, the present invention provides synthetic high density lipoprotein (HDL) complexes of therapeutic agents that are effective nanoparticle delivery vehicles for therapeutic agents (e.g., poorly water-soluble therapeutic agents).

Plasma lipoproteins are composed of lipid and protein components that form a globular complex designed to transport water-insoluble lipids in a physiological environment. The two phase structure of lipoproteins includes an outer shell made up of amphiphilic components (phospholipid and protein components) and an interior core containing highly hydrophobic lipids. This two phase structure allows lipoproteins to fulfill their roles as drug delivery agents, particularly in the transport of water-insoluble drugs.

The present invention provides delivery vehicles are provided in a formulation of a therapeutic agent that is encapsulated in a synthetic self-assembled nanoparticle that includes a lipid binding protein and a lipid monolayer. The interior of the particle represents a hydrophobic core region where therapeutic agents (e.g., poorly water-soluble therapeutic agents) may be incorporated. In contrast to liposomes, which include an aqueous interior core surrounded by a phospholipid bilayer, the nanoparticle vehicle of the invention is composed of a shell (e.g., monolayer) surrounding a hydrophobic interior (i.e., core).

The hydrophobic nature of the interior of the synthetic HDL particle of the invention allows the encapsulation of hydrophobic molecules, in a manner similar to the native core component of HDL (cholesteryl esters). The character of those compounds that are appropriate candidates for encapsulation, including chemotherapeutic agents, can be defined by their octanol/water partition coefficient X log P (Wang et al. Chem. Inf Comput. Sci. 1997, 37, 615-621). For example, the coefficient for paclitaxel is 3.0. In the practice of the invention, therapeutic agents with X log P greater than 2.0 are excellent candidates for incorporation into the vehicle of the invention. This characteristic includes over half of the approved pharmaceutical agents currently employed for parenteral administration. As used herein, the term “poorly water-soluble therapeutic agent” refers to a therapeutic agent having an octanol/water partition coefficient X log P greater than 4.0.

In one aspect, the invention provides a synthetic self-assembled nanoparticle that includes a lipid layer (e.g., monolayer) comprising an amphiphilic lipid (e.g., a phosphatidylcholine) and one or more therapeutic agents.

As used herein, the term “self-assembly” (“self-assembled” or “self-assembling”) refers to the formation of synthetic HDL nanoparticles from ingredients (such as lipids and relatively low molecular weight proteins, such as apolipoprotein (e.g., apolipoprotein A1, also referred to herein as Apo-A1 or ApoA-I) assembled into a particle of greater molecular weight without the application of a physical force, such as sonication, high pressure, membrane intrusion, or centrifugation.

The core (i.e., interior) of the particle is a hydrophobic region where the transported materials reside in a manner similar to the native cholesteryl esters in HDL. Particles of the invention do not include a hydrophilic or aqueous core.

The shell (e.g., lipid monolayer) of the particle includes lipids (e.g., phospholipids) with their polar head groups facing away from the interior of the particle. Any suitable lipid may be used that along with a lipid binding protein to provide the scaffolding for the substantially spherical particle to accommodate the drug to be transported in the interior of the particle. The term “monolayer-forming lipid” refers to a compound that is capable of forming a lipid monolayer serving as an outer shell of the basic lipoprotein structure. In some embodiments, the lipid shell is composed of phospholipids, such as phosphatidylcholines.

As used herein, the term “lipid binding protein” refers to synthetic or naturally occurring peptides or proteins that are able to sustain a stable complex with lipid surfaces and thus able to function to stabilize the lipid shell the nanoparticle of the invention. The nanoparticles of the invention may include one or more types of lipid binding proteins or apolipoproteins that are natural components of plasma lipoproteins. In some embodiments, nanoparticles can be prepared using small synthetic peptides that may serve as surrogates for native lipoproteins. Apolipoproteins generally include a high content of amphipathic alpha-helix motif that facilitates their ability to bind to hydrophobic surfaces, including lipids. An important characteristic of apolipoproteins is to support the structure of monolayers, vesicles or bilayers, composed primarily of phospholipids and to transform them into disc-shaped complexes. Under physiological conditions, the discoidal complexes undergo a transition to a spherical structure.

The present invention provides nanoparticle delivery vehicles, methods for using the nanoparticle delivery vehicles, and methods for making the nanoparticle delivery vehicles.

Nanoparticle Delivery Vehicles

In one aspect, the invention provides high density lipoprotein (HDL) nanoparticle delivery vehicles. On administration, the nanoparticle delivery vehicles of the invention are stable in the circulatory system. As used herein, the term “stable in the circulatory system” refers to the ability of the vehicle to substantially maintain its therapeutic agent content while in the circulatory system. In various embodiments, the vehicle maintains greater than 80 percent, greater than 85 percent, greater than 90 percent, greater than 95 percent, or greater than 99 percent of the therapeutic agent loaded into the nanoparticle delivery vehicle. The nanoparticle delivery vehicles substantially maintain their therapeutic agent content by increasing the hydrophobicity of the portion of the vehicle responsible for therapeutic agent transport. The increase in the vehicle hydrophobicity is effective to increase stability of poorly water-soluble therapeutic agents (e.g., therapeutic agents having an octanol/water partition coefficient X log P greater than 4.0) in the vehicle.

In certain embodiments, the core (e.g., interior) of the nanoparticle has increased hydrophobicity compared to native HDL particles. For these embodiments, the hydrophobicity of one or more lipid components of the vehicle's core is increased relative to the hydrophobicity of the component in native HDL. In certain embodiments, the shell (e.g., lipid monolayer) of the nanoparticle has increased hydrophobicity compared to native HDL particles. For these embodiments, the hydrophobicity of one or more phospholipids of the vehicle's shell is increased relative to the hydrophobicity of the component in native HDL. In certain embodiments, the hydrophobicity of the core and the shell of the vehicle are increased compared to native HDL. In other embodiments, the core and/or shell of the vehicle can further include components not found in native HDL particles that are effective to increase the hydrophobicity of the vehicle's core and/or shell.

The hydrophobicity of the vehicle can be increased by a variety of methods including increasing the hydrophobicity of HDL components by, for example, increasing the number of carbon atoms in the fatty acid chains of the component phospholipids or cholesteryl esters. In any event, the vehicles of the invention substantially maintain their therapeutic agent content in the circulatory system (at concentrations below the solubility limit of the therapeutic agent, the therapeutic agent remains in the vehicle).

In certain embodiments, the invention provides a nanoparticle delivery vehicle, comprising a high density lipoprotein complex. The complex comprises:

(a) a hydrophobic core having increased hydrophobicity compared to native high density lipoprotein, the core comprising
(i) a lipid component, and
(ii) a therapeutic agent (e.g., a poorly water-soluble therapeutic agent), and

(b) a shell (e.g., a hydrophilic shell) surrounding the core, the shell comprising a phospholipid.

The vehicle's core is a substantially non-aqueous environment. On administration, the vehicle's shell is in contact with the aqueous environment of the circulatory system.

In certain embodiments, the lipid component includes a lipid binding protein. Representative lipid binding proteins include apolipoprotein A1 (ApoA-1).

In certain embodiments, the lipid component includes cholesterol. In certain embodiments, the lipid component includes one or more cholesteryl esters as described above, including cholesterol fatty acid esters. Representative cholesterol fatty acid esters include cholesteryl laurate, cholesteryl myristate, cholesteryl palmitate, cholesteryl stearate, and cholesteryl oleate.

In certain embodiments, the lipid component includes one or more of a sphingomyelin, a cationic phospholipid, and a glycolipid.

In certain embodiments, the lipid component includes a phosphatidylcholine. Representative phosphatidylcholines include dimyristoylphosphatidylcholine (DMPC), dioleoylphosphatidylcholine (DOPC), dipalmitoylphosphatidylcholine (DPPC), egg yolk phosphatidylcholine (egg PC), soybean phosphatidylcholine, and mixtures thereof.

In certain embodiments, the lipid component includes a mixture of a phosphatidylcholine, cholesterol, and a cholesterol fatty acid ester.

The vehicle of the invention includes a therapeutic agent (e.g., a poorly water-soluble therapeutic agent). The nature of the therapeutic agent effectively delivered by the vehicle of the invention is not particularly critical so long as the therapeutic agent is advantageously retained in the vehicle while the vehicle is in the circulatory system (e.g., poorly water soluble therapeutic agent). Effectively delivered therapeutic agents (e.g., poorly water-soluble therapeutic agents) include anti-inflammatory agents (i.e., therapeutic agents effective for treating inflammation and inflammatory diseases), therapeutic agents effective for treating atherosclerosis, and chemotherapeutic agents. A representative chemotherapeutic agent is paclitaxel.

The vehicle (e.g., vehicle shell) includes one or more phospholipids. Suitable phospholipids include those described above with regard to the nanoparticles of the invention.

In certain embodiments, one or more of the shell's phospholipid has increased hydrophobicity compared to phospholipids in native high density lipoprotein. Suitable phospholipids include phosphatidylcholines, phosphatidylethanolamines, phosphatidylglycerols, phosphatidylserines, phosphatidylinositols, and phosphatidic acids.

In certain embodiments, the phospholipid is a diacylphosphatidylcholine. Representative diacylphosphatidylcholines include distearoylphosphatidylcholine (DSPC), dioleoylphosphatidylcholine (DOPC), dipalmitoylphosphatidylcholine (DPPC), dilinoleoylphosphatidylcholine DLPC), palmitoyloleoylphosphatidylcholine (POPC), palmitoyllinoleoylphosphatidylcholine, stearoyllinoleoylphosphatidylcholine stearoyloleoylphosphatidylcholine, stearoylarachidoylphosphatidylcholine, didecanoylphosphatidylcholine (DDPC), dierucoylphosphatidylcholine (DEPC), dilinoleoylphosphatidylcholine (DLOPC), dimyristoylphosphatidylcholine (DMPC), myristoylpalmitoylphosphatidylcholine (MPPC), myristoylstearoylphosphatidylcholine (MSPC), stearoylmyristoylphosphatidylcholine (SMPC), palmitoylmyristoylphosphatidylcholine (PMPC), palmitoylstearoylphosphatidylcholine (PSPC), stearoylpalmitoylphosphatidylcholine (SPPC), and stearoyloleoylphosphatidylcholine (SOPC).

Representative therapeutic agents advantageously delivered by the synthetic high density lipoprotein (HDL) complex of the invention include the representative therapeutic agent described above for the nanoparticle of the invention, including antineoplastic agents (e.g., paclitaxel) and immunosuppressive agents.

In certain embodiments, the vehicle has substantially the same electrophoretic mobility, size, and chemical composition as a native high density lipoprotein. In certain embodiments, the vehicle has a size from about 5 to about 100 nm. In certain embodiments, the vehicle has a molecular weight of about 120 k to about 500 k Dalton. In certain embodiments, the vehicle is spherical, oval, or discoidal in shape.

In a related aspect, the invention provides pharmaceutical compositions that include a nanoparticle delivery vehicle and a pharmaceutically acceptable carrier. The pharmaceutically acceptable carrier is selected based on the mode of vehicle administration. Suitable carriers for intravenous administration include saline and dextrose solutions known in the art.

Methods of Nanoparticle Delivery Vehicle Use

In another aspect of the invention, methods for using the nanoparticle delivery vehicle are provided.

In one embodiment, the invention provides a method for delivering a therapeutic agent (e.g., a poorly water-soluble therapeutic agent) to a subject. In the method, a nanoparticle delivery vehicle of the invention is administered to a subject.

In another embodiment, the invention provides a method for the treatment of cancer. In the method, a therapeutically effective of a nanoparticle delivery vehicle of the invention is administered to a subject in need thereof. In this method, the cancer is one treatable by administration of the specific therapeutic agent (e.g., a poorly water-soluble therapeutic agent). In certain embodiments, the cancer is prostate cancer, ovarian cancer, or breast cancer. In certain embodiments of the methods of the invention, the therapeutic agent is a chemotherapeutic agent. In certain embodiments, the therapeutic agent is paclitaxel.

In further embodiments, the invention provides methods for the treatment of inflammatory diseases and methods for treatment of atherosclerosis. In these methods, a therapeutically effective of a nanoparticle delivery vehicle of the invention is administered to a subject in need thereof. In these methods, the therapeutic agent is an anti-inflammatory agent or an agent effective for treating atherosclerosis, respectively.

Representative conditions and disease treatable by the synthetic high density lipoprotein (HDL) complexes of the invention include the cancers described above treatable by the nanoparticles of the invention.

In certain embodiments of the methods of the invention, an effective amount of the vehicle is delivered systemically to a subject in need thereof. The vehicle is suitably administered parenterally, intravenously, intramuscularly, subcutaneously, transmucosally, or transdermally. As used herein, the term “subject” refers to any cell, animal tissue, or a vertebrate animal. In some embodiments, the individual is a vertebrate, such as a human, a nonhuman primate, or an experimental animal, such as a mouse or rat. In some embodiments, particles are formulated as a carrier, suitable for administration to a subject. The term “effective amount” refers to the amount of a therapeutic agent sufficient to bring about the desired results in an experimental setting. A “therapeutically effective amount” or “therapeutic dose” refers to an amount of a therapeutic agent that is sufficient to produce beneficial clinical results, such as reduction in tumor size or remission for cancer patients.

Methods for Making Nanoparticle Delivery Vehicles

In a further aspect, the invention provides a method for making the nanoparticle delivery vehicle. In certain embodiments, the method comprises:

(a) mixing lipid components in a suitable organic solvent to give a lipid mixture;

(b) adding a therapeutic agent (e.g., a poorly water-soluble therapeutic agent) to the lipid mixture to provide a lipid-therapeutic agent mixture;

(c) drying the lipid-therapeutic agent mixture under nitrogen to provide a solid;

(d) dispersing the solid in an aqueous solution to provide a dispersed mixture;

(e) mixing the dispersed mixture in a buffer to provide a buffered mixture;

(f) adding a suitable salt to the buffered mixture to provide a salt mixture;

(g) adding a lipid binding protein to the salt mixture to provide a lipid binding protein mixture;

(h) incubating the lipid binding protein mixture to provide an incubated mixture; and

(i) subjecting the incubated mixture to dialysis with one or more buffer changes to facilitate the self-assembly and formation of the nanoparticle delivery vehicle.

In certain embodiments, the lipid components include a phosphatidylcholine, cholesterol, and a cholesterol ester.

In certain embodiments, the organic solvent is dimethylsulfoxide.

In certain embodiments, the therapeutic agent is a chemotherapeutic agent. In one embodiment, the therapeutic agent is paclitaxel.

In certain embodiments, the aqueous solution is 3% dimethylsulfoxide.

In certain embodiments, the salt is sodium cholate.

In certain embodiments, the lipid binding protein is apolipoprotein A1.

The preparation of a representative nanoparticle delivery vehicle of the invention that includes paclitaxel is described in Example 1.

The following example is provided for the purpose of illustrating, not limiting, the invention.

### Preparation of a Representative Nanoparticle Delivery Vehicle: HDL/Paclitaxel Particles

Recombinant ApoA-1 is prepared as described in Ryan et al. (2003) Protein Expression and Purification 27:98-103, and can be used to prepare representative HDL/paclitaxel complexes of the invention.

The particles are prepared by a process involving cholate dialysis as described below to produce a spherical structure with paclitaxel in the interior hydrophobic core region. The lipid mixture (egg yolk phosphatidylcholine [PC], cholesterol and cholesteryl oleate in the ratio of 3.8:1:88.5) and 2 mg paclitaxel is dried under nitrogen to provide a thin film, which is then dispersed in dimethylsulfoxide and subsequently in 1.4 ml of 10 mM Tris, 0.1 M KCl, 1 mM EDTA, pH 8.0). Sodium cholate, 140 μl (100 mg/ml stock in [0.15 M NaCl 0.003 M KCl, 0.15 M KH₂PO₄, pH 7.4, designated as PBS]) is added to produce mixtures with a final PC to cholate molar ratio of about 1:1.6. ApoA-I (12.7 mg/ml) in 0.4 ml of PBS is added to this mixture and the final volume is adjusted to 2 ml with PBS. The lipid/protein/cholate mixture is then incubated for 12 hrs at 4° C., followed by dialysis (2 liter of PBS, for two days) with three buffer changes.

While the preferred embodiment of the invention has been illustrated and described, it will be appreciated that various changes can be made therein without departing from the spirit and scope of the invention.

1. A phospholipid-coated therapeutic agent nanoparticle, comprising a particulate therapeutic agent coated with a cholesteryl ester and one or more phospholipids,
wherein the nanoparticle is stable in aqueous delivery vehicles for administration and releases the therapeutic agent substantially instantaneously upon exposure to physiological fluid.

2. A phospholipid-coated therapeutic agent nanoparticle, comprising a particulate therapeutic agent coated with a cholesteryl ester and one or more phospholipids,
wherein the nanoparticle is as stable in aqueous delivery vehicles for administration as synthetic polymeric micelles containing a therapeutic agent (Genexol-PM®) and is as effective in releasing the therapeutic agent under physiological conditions as a human-serum albumin-coated therapeutic agent (Abraxane®).

3. A phospholipid-coated therapeutic agent nanoparticle, comprising a particulate therapeutic agent coated with a cholesteryl ester and one or more phospholipids,
wherein the phospholipid is selected from the group consisting of a mono-acylphospholipid, a diacylphospholipid, and mixtures thereof.

4. A phospholipid-coated therapeutic agent nanoparticle, consisting essentially of a particulate therapeutic agent coated with a cholesteryl ester and one or more phospholipids.

5. A phospholipid-coated therapeutic agent nanoparticle, consisting of a particulate therapeutic agent coated with a cholesteryl ester and one or more phospholipids.

6. The nanoparticle of any one of claim 1-5, wherein the cholesteryl ester has a fatty acid component having from 10 to 22 carbons.

7. The nanoparticle of any one of claim 1-5, wherein the cholesteryl ester has a fatty acid component having from 12 to 20 carbons.

8. The nanoparticle of any one of claim 1-5, wherein the cholesteryl ester has a fatty acid component having from 16 to 18 carbons.

9. The nanoparticle of any one of claim 1-5, wherein the ratio of cholesteryl ester to phospholipid is from about 1:1000 to about 1:10 w/w.

10. The nanoparticle of any one of claim 1-5, wherein the ratio of cholesteryl ester to phospholipid is from about 1:200 to about 1:50 w/w.

11. The nanoparticle of any one of claim 1-5, wherein the phospholipid is a diacylphospholipid.

12. The nanoparticle of any one of claim 1-5, wherein the phospholipid is a diacylphospholipid selected from the group consisting of diacylphosphatidylcholines, diacylphosphatidylethanolamines, diacylphosphatidylglycerols, diacylphosphatidylserines, diacylphosphatidylinositols, and diacylphosphatidic acids, and mixtures thereof.

13. The nanoparticle of any one of claim 1-5, wherein the phospholipid is selected from the group consisting of phosphatidylcholines, phosphatidylethanolamines, phosphatidylglycerols, phosphatidylserines, phosphatidylinositols, and phosphatidic acids, and their lysophosphatidyl and diacyl phospholipid counterparts.

14. The nanoparticle of any one of claim 1-5, wherein the phospholipid is a phosphatidylcholine.

15. The nanoparticle of any one of claim 1-5, wherein the phospholipid is a phosphatidylcholine having a fatty acid component having from 10 to 22 carbons.

16. The nanoparticle of any one of claim 1-5, wherein the phospholipid is a phosphatidylcholine having a fatty acid component having from 10 to 12 carbons.

17. The nanoparticle of any one of claim 1-5, wherein the phospholipid is a diacylphosphatidylcholine selected from the group consisting of distearoylphosphatidylcholine (DSPC), dioleoylphosphatidylcholine (DOPC), dipalmitoylphosphatidylcholine (DPPC), dilinoleoylphosphatidylcholine DLPC), palmitoyloleoylphosphatidylcholine (POPC), palmitoyllinoleoylphosphatidylcholine, stearoyllinoleoylphosphatidylcholine stearoyloleoylphosphatidylcholine, stearoylarachidoylphosphatidylcholine, didecanoylphosphatidylcholine (DDPC), dierucoylphosphatidylcholine (DEPC), dilinoleoylphosphatidylcholine (DLOPC), dimyristoylphosphatidylcholine (DMPC), myristoylpalmitoylphosphatidylcholine (MPPC), myristoylstearoylphosphatidylcholine (MSPC), stearoylmyristoylphosphatidylcholine (SMPC), palmitoylmyristoylphosphatidylcholine (PMPC), palmitoylstearoylphosphatidylcholine (PSPC), stearoylpalmitoylphosphatidylcholine (SPPC), stearoyloleoylphosphatidylcholine (SOPC), and their diacyl lyso-phosphatidylcholine counterparts.

18. The nanoparticle of any one of claim 1-5, wherein the phospholipid is a mono-acylphospholipid.

19. The nanoparticle of any one of claim 1-5, wherein the phospholipid is a mono-acylphospholipid selected from the group consisting of lysophosphatidylcholines, lysophosphatidylethanolamines, lysophosphatidylglycerols, lysophosphatidylserines, lysophosphatidylinositols, and lysophosphatidic acids, and mixtures thereof.

20. The nanoparticle of any one of claim 1-5, wherein the phospholipid is a lysophosphatidylcholine having a fatty acid component having from 10 to 22 carbons.

21. The nanoparticle of any one of claim 1-5, wherein the phospholipid is a lysophosphatidylcholine having a fatty acid component having from 10 to 12 carbons.

22. The nanoparticle of any one of claim 1-5, wherein the phospholipid is a combination of a phosphatidylcholine and a lysophosphatidylcholine.

23. The nanoparticle of claim 22, wherein the ratio of diacylphospholipid to mono-acylphospholipid from about 90:10 to 60:40 w/w percent.

24. The nanoparticle of claim 22, wherein the ratio of diacylphospholipid to mono-acylphospholipid of about 80:20 w/w percent.

25. The nanoparticle of any one of claim 1-5, wherein the therapeutic agent is therapeutic agent having an X log P greater than 2.0.

26. The nanoparticle of any one of claim 1-5, wherein the therapeutic agent is selected from the group consisting of analgesics/antipyretics, anesthetics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, anti-infectives, bronchodialators, hormones, hypoglycemic agents, hypolipidemic agents, proteins, nucleic acids, agents useful for erythropoiesis stimulation, antiulcer/antireflux agents, antinauseants/antiemetics, and oil-soluble vitamins.

27. The nanoparticle of any one of claim 1-5, wherein the therapeutic agent is an antineoplastic selected from adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferon, camptothecin and derivatives thereof, phenesterine, paclitaxel and derivatives thereof, docetaxel and derivatives thereof, epothilones and derivatives thereof, vinblastine, vincristine, tamoxifen, etoposide, or piposulfan.

28. The nanoparticle of any one of claim 1-5, wherein the therapeutic agent is paclitaxel or derivatives thereof.

29. The nanoparticle of any one of claim 1-5, wherein the therapeutic agent is in crystalline form.

30. The nanoparticle of any one of claim 1-5, wherein the therapeutic agent is in amorphous form.

31. The nanoparticle of any one of claim 1-5 having an average diameter from about 30-300 nm.

32. The nanoparticle of any one of claim 1-5 having an average diameter from about 80-200 nm.

33. A pharmaceutical composition, comprising the nanoparticle of any one of claim 1-32.

34. The pharmaceutical composition of claim 33, wherein the nanoparticle is in the form of a dry powder.

35. A pharmaceutical composition, comprising the nanoparticle of any one of claim 1-32 and a pharmaceutically acceptable carrier.

36. The pharmaceutical composition of claim 35, wherein the nanoparticle is stably suspended in an aqueous medium.

37. The pharmaceutical composition of claim 35, wherein the composition further comprises a particle size stabilizing agent.

38. A pharmaceutical composition for injection, comprising the nanoparticle of any one of claim 1-32 and a pharmaceutically acceptable carrier.

39. A unit dosage form for treating in an individual, comprising the nanoparticle of any one of claim 1-32 and a pharmaceutically acceptable carrier.

40. A kit, comprising a container comprising the nanoparticle of any one of claim 1-32, a container comprising a pharmaceutically acceptable carrier for reconstituting the nanoparticle, and instructions for using the kit in treating a disease or condition.

41. A kit, comprising a container comprising the nanoparticle of any one of claim 1-32 suspended in a pharmaceutically acceptable carrier, and instructions for using the kit in treating a disease or condition.

42. A method of treating a disease or condition in an individual, comprising administering to an individual in need thereof an effective amount of the nanoparticle of anyone of claim 1-32.

43. The method of claim 42, wherein the disease or condition is a proliferative disease or condition.

44. The method of claim 42, wherein the therapeutic agent is paclitaxel and the disease is a disease treatable by administering paclitaxel.

45. The method of claim 42, wherein the therapeutic agent is paclitaxel and the disease is a cancer treatable by administering paclitaxel.

46. A method for preparation of a phospholipid-coated therapeutic agent nanoparticle, comprising:
subjecting an organic phase containing a therapeutic agent dispersed therein and aqueous medium containing phospholipid to high shear conditions in a high pressure homogenizer to provide a homogenized phospholipid-coated therapeutic agent nanoparticle mixture.

47. The method of claim 46, wherein subjecting to high shear conditions comprises using a high pressure homogenizer at a pressure in the range of about 3,000 up to 30,000 psi.

48. The method of claim 46 further comprising removing the organic phase from the mixture.

49. The method of claim 46 further comprising removing the aqueous medium from the mixture.

50. The method of claim 46, wherein the organic phase comprises a mixture of a substantially water immiscible organic solvent and a water soluble organic solvent.

51. The method of claim 46, wherein said aqueous medium is selected from water, buffered aqueous media, saline, buffered saline, solutions of amino acids, solutions of sugars, solutions of vitamins, solutions of carbohydrates, or combinations thereof.

52. The method of claim 46, wherein the particles have an average diameter of from about 30 nm to about 300 nm.

53. The method of claim 46 further comprising sterile filtering the mixture.

54. The method of claim 46, wherein the nanoparticle is a nanoparticle of any one of claim 1-32.

55. The method of claim 49, wherein removing the aqueous medium from the mixture comprises lyophilizing the mixture to provide a nanoparticle powder.

56. The method of claim 55, wherein the nanoparticle is a nanoparticle of any one of claim 1-32.

57. A method for preparation of a phospholipid-coated therapeutic agent nanoparticle, comprising:
dissolving a therapeutic agent and a phospholipid in an organic phase to provide a solution;
concentrating the solution to dryness to provide a film; and
hydrating the film with water to provide a aqueous suspension of phospholipid-coated therapeutic agent nanoparticles.

58. The method of claim 57, wherein the organic phase comprises ethanol.

59. The method of claim 57, wherein concentrating the solution to dryness comprises rotary evaporation.

60. The method of claim 57, wherein the water is deionized water.

61. The method of claim 57, wherein the particles have an average diameter of from about 30 nm to about 300 nm.

62. The method of claim 57 further comprising sterile filtering the aqueous suspension.

63. The method of claim 57, wherein the nanoparticle is a nanoparticle of any one of claim 1-32.

64. A nanoparticle delivery vehicle, comprising a high density lipoprotein complex comprising:
(a) a hydrophobic core having increased hydrophobicity compared to native high density lipoprotein, the core comprising
(i) a lipid component, and
(ii) a therapeutic agent, and,
(b) a shell surrounding the core, the shell comprising a phospholipid.

65. The vehicle of claim 64, wherein the lipid component comprises apolipoprotein A1 (ApoA-1).

66. The vehicle of claim 64, wherein the lipid component comprises cholesterol.

67. The vehicle of claim 64, wherein the lipid component comprises one or more cholesterol fatty acid esters.

68. The vehicle of claim 64, wherein the lipid component comprises a cholesterol fatty acid ester selected from the group consisting of cholesteryl laurate, cholesteryl myristate, cholesteryl palmitate, cholesteryl stearate, cholesteryl oleate, and mixtures thereof.

69. The vehicle of claim 64, wherein the lipid component comprises one or more of a sphingomyelin, a cationic phospholipid, or a glycolipid.

70. The vehicle of claim 64, wherein the lipid component comprises a phosphatidylcholine.

71. The vehicle of claim 64, wherein the lipid component comprises a phosphatidylcholine selected from the group consisting of dimyristoylphosphatidylcholine (DMPC), dioleoylphosphatidylcholine (DOPC), dipalmitoylphosphatidylcholine (DPPC), egg yolk phosphatidylcholine (egg PC), soybean phosphatidylcholine, and mixtures thereof.

72. The vehicle of claim 64, wherein the lipid component comprises a mixture of a phosphatidylcholine, cholesterol, and a cholesterol fatty acid ester.

73. The vehicle of claim 64, wherein the therapeutic agent is a poorly water-soluble therapeutic agent.

74. The vehicle of claim 64, wherein the therapeutic agent is a chemotherapeutic agent.

75. The vehicle of claim 64, wherein the therapeutic agent is paclitaxel.

76. The vehicle of claim 64, wherein the phospholipid has increased hydrophobicity compared to phospholipids in native high density lipoprotein.

77. The vehicle of claim 64, wherein the phospholipid is selected from the group consisting of phosphatidylcholines, phosphatidylethanolamines, phosphatidylglycerols, phosphatidylserines, phosphatidylinositols, phosphatidic acids, and mixtures thereof.

78. The vehicle of claim 64, wherein the phospholipid is a diacylphosphatidylcholine selected from the group consisting of distearoylphosphatidylcholine (DSPC), dioleoylphosphatidylcholine (DOPC), dipalmitoylphosphatidylcholine (DPPC), dilinoleoylphosphatidylcholine DLPC), palmitoyloleoylphosphatidylcholine (POPC), palmitoyllinoleoylphosphatidylcholine, stearoyllinoleoylphosphatidylcholine stearoyloleoylphosphatidylcholine, stearoylarachidoylphosphatidylcholine, didecanoylphosphatidylcholine (DDPC), dierucoylphosphatidylcholine (DEPC), dilinoleoylphosphatidylcholine (DLOPC), dimyristoylphosphatidylcholine (DMPC), myristoylpalmitoylphosphatidylcholine (MPPC), myristoylstearoylphosphatidylcholine (MSPC), stearoylmyristoylphosphatidylcholine (SMPC), palmitoylmyristoylphosphatidylcholine (PMPC), palmitoylstearoylphosphatidylcholine (PSPC), stearoylpalmitoylphosphatidylcholine (SPPC), stearoyloleoylphosphatidylcholine (SOPC), and mixtures thereof.

79. The vehicle of claim 64, wherein the core is a substantially non-aqueous environment.

80. The vehicle of claim 64, wherein the vehicle has a size from about 5 to about 100 nm.

81. The vehicle of claim 64, wherein the vehicle is spherical, oval, or discoidal in shape.

82. The vehicle of claim 64, wherein the vehicle has a molecular weight of about 120 k to about 500 k Dalton.

83. The vehicle of claim 64, wherein the vehicle has approximately the same electrophoretic mobility, size, and chemical composition as a native high density lipoprotein.

84. A method for delivering a therapeutic agent to a subject, comprising administering the nanoparticle delivery vehicle of any one of claim 64-83 to a subject.

85. The method of claim 84, wherein the therapeutic agent is a chemotherapeutic agent.

86. The method of claim 84, wherein the therapeutic agent is paclitaxel.

87. The method of claim 84, wherein the vehicle is delivered parenterally, intravenously, intramuscularly, subcutaneously, transmucosally, or transdermally.

88. A method for the treatment of cancer in a subject, comprising administering a therapeutically effective amount of the nanoparticle delivery vehicle of any of claims 64-83 to a subject in need thereof, wherein the cancer is treatable by the therapeutic agent.

89. The method of claim 88, wherein the therapeutic agent is a chemotherapeutic agent.

90. The method of claim 88, wherein the therapeutic agent is paclitaxel.

91. The method of claim 88, wherein the vehicle is delivered parenterally, intravenously, intramuscularly, subcutaneously, transmucosally, or transdermally.

92. The method of claim 88, wherein the cancer is prostate cancer, ovarian cancer, or breast cancer.

93. A method for the treatment of an inflammatory disease in a subject, comprising administering a therapeutically effective amount of the nanoparticle delivery vehicle of any of claims 64-83 to a subject in need thereof, wherein the inflammatory disease is treatable by the therapeutic agent.

94. A method for the treatment of atherosclerosis in a subject, comprising administering a therapeutically effective amount of the nanoparticle delivery vehicle of any of claim 64-83 to a subject in need thereof, wherein atherosclerosis is treatable by the therapeutic agent.

95. A method for making a nanoparticle delivery vehicle, comprising:
(a) mixing lipid components in a suitable organic solvent to give a lipid mixture;
(b) adding a therapeutic agent to the lipid mixture to provide a lipid-therapeutic agent mixture;
(c) drying the lipid-therapeutic agent mixture under nitrogen to provide a solid;
(d) dispersing the solid in an aqueous solution to provide a dispersed mixture;
(e) mixing the dispersed mixture in a buffer to provide a buffered mixture;
(f) adding a suitable salt to the buffered mixture to provide a salt mixture;
(g) adding a lipid binding protein to the salt mixture to provide a lipid binding protein mixture;
(h) incubating the lipid binding protein mixture to provide an incubated mixture; and
(i) subjecting the incubated mixture to dialysis with one or more buffer changes to facilitate the self-assembly and formation of a nanoparticle delivery vehicle.

96. The method of claim 95, wherein the lipid components comprises a phosphatidylcholine, cholesterol, and a cholesterol ester.

97. The method of claim 95, wherein the organic solvent is dimethylsulfoxide.

98. The vehicle of claim 95, wherein the therapeutic agent is a poorly water-soluble therapeutic agent.

99. The method of claim 95, wherein the therapeutic agent is a chemotherapeutic agent.

100. The method of claim 95, wherein the therapeutic agent is paclitaxel.

101. The method of claim 95, wherein the aqueous solution is 3% dimethylsulfoxide.

102. The method of claim 95, wherein the salt is sodium cholate.

103. The method of claim 95, wherein the lipid binding protein is apolipoprotein A1.

104. The method of claim 95, wherein the nanoparticle delivery vehicle is any one of claim 64-83.

105. A method for treating cancer in a subject by targeting cancer cells expressing a high density lipoprotein receptor, comprising:
administering to a subject in need thereof a therapeutically effective amount of a nanoparticle delivery vehicle of any one of claim 64-83, whereby the therapeutic agent is transferred to an endogenous plasma high density lipoprotein to provide a high density lipoprotein particle containing the therapeutic agent;
wherein the high density lipoprotein particle containing the therapeutic agent associate with a cancer cell expressing a high density lipoprotein receptor, whereby the therapeutic agent is delivered to the cancer cell.

106. The method of claim 105, wherein the high density lipoprotein receptor is a scavenger receptor type B1 (SR-B1).

107. The method of claim 105, wherein administering the composition comprises systemic delivery.

108. The method of claim 105, wherein administering the composition comprises intravenous injection.

109. The vehicle of claim 105, wherein the therapeutic agent is a poorly water-soluble therapeutic agent.

110. The method of claim 105, wherein the therapeutic agent is a chemotherapeutic agent.

111. The method of claim 105, wherein the therapeutic agent is paclitaxel.
